(19) United States
(12) Patent Application Publication
(10) Pub. No.: US 2008/0139536 A1
(43) Pub. Date: Jun. 12, 2008

(54) Benzodiazepines for Treating or Preventing Rsv Infection

(75) Inventors: Verity Dowdell, London (GB); Richard Kelsey, London (GB); Malcolm Carter, London (GB); Dagmar Alber, London (GB); Elisa Henderson, London (GB)

Correspondence Address: LAHIVE & COCKFIELD, LLP, ONE POST OFFICE SQUARE, BOSTON, MA, 02109-2127, US

(73) Assignee: Novartis Pharmaceuticals Corporation, Cambridge, MA (US)

(21) Appl. No.: 10/593,667

(22) PCT Filed: Mar. 18, 2005
(86) PCT No.: PCT/GB2005/001023

§ 371 Date: Aug. 2, 2007

(30) Foreign Application Priority Data
Mar. 19, 2004 (GB) 0406280.8

(51) Int. Cl. A61K 31/5513; C07D 243/24; A61P 31/12

(52) U.S. Cl. 514/221; 540/509

(57) ABSTRACT

Use of a compound which is (a) a benzodiazepine derivative of the formula (I) or an N-oxide thereof or (b) a pharmaceutically acceptable salt thereof, in the manufacture of a medicament for use in treating or preventing an RSV infection wherein: —R¹ represents C₁₋₆ alkyl, aryl or heteroaryl; —R² represents hydrogen or C₁₋₆ alkyl; each R³ is the same or different and represents halogen, hydroxy, C₁₋₆ alkyl, C₁₋₆ alkoxy, C₁₋₆ alkylthio, C₁₋₆ haloalkyl, C₁₋₆ haloalkoxy, amino, mono(C₁₋₆ alkyl)amino, di(C₁₋₆alkyl)amino, vitro, cyano, —CO₂R′, —CONR′R″, —NH—CO—R′, —S(O)R′, —S(O)₂R′, —NH—S(O)₂R′, —S(O)NR′R″ or —S(O)₂NR′R″, wherein each R′ and R″ is the same or different and represents hydrogen or C₁₋₆ alkyl; n is from O to 3; R⁴ represents hydrogen or C₁₋₆ alkyl; X represents —CO—, —CO—NR′—, —S(O)— or —S(O)₂—, wherein R′ is hydrogen or a C₁-C₆ alkyl group; and R⁵ represents a heteroaryl or heterocyclyl group which is substituted by a C1C6 hydroxyalkyl group or a —(C₁-C₄ alkyl)-X₁—(C₁-C₄ alkyl)-X₂—(C₁-C₄ alkyl} group, wherein X₁ represents -0-, —S— or —NR′—, wherein R′ represents H or a C₁-C₄ alkyl group and X₂ represents —CO—, —SO— or —SO₂—, or R₅ represents -A₁-Y-A₂, wherein: -A₁ is an aryl, heteroaryl, carbocyclyl or heterocyclyl group; 25 —Y represents a direct bond or a C₁-C₄ alkylene, —SO₂—, —CO—, -0-, —S— or —NR′-moiety, wherein R′ is a C₁-C₆ alkyl group; and -A₂ is an aryl, heteroaryl, carbocyclyl or heterocyclyl group.

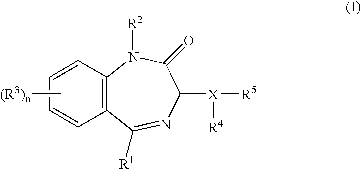

The present invention relates to a series of benzodiazepine derivatives which are active against Respiratory Syncytial Virus (RSV).

RSV is a major cause of respiratory illness in patients of all ages. In adults, it tends to cause mild cold symptoms. In school-aged children, it can cause a cold and bronchial cough. In infants and toddlers it can cause bronchiolitis (inflammation of the smaller airways of the lungs) or pneumonia. It has also been found to be a frequent cause of middle ear infections (otitis media) in pre-school children. RSV infection in the first year of life has been implicated in the development of asthma during childhood.

Current anti-RSV therapy involves the use of a monoclonal antibody to RSV, called palivizumab. Such use of palivizumab is a prophylactic, rather than therapeutic, treatment of RSV. However, although this antibody is often effective, it is expensive. Indeed, its expense means that it is unavailable for many people in need of anti-RSV therapy. There is therefore an urgent need for effective alternatives to existing anti-RSV therapy.

It has now surprisingly been found that the particular benzodiazepine derivatives of the general formula (I) set out below are active against RSV.

Accordingly, the present invention provides, in a first embodiment, the use of a compound which is (a) a benzodiazepine derivative of formula (I) or an N-oxide thereof, or (b) a pharmaceutically acceptable salt thereof, in the manufacture of a medicament for use in treating or preventing an RSV infection

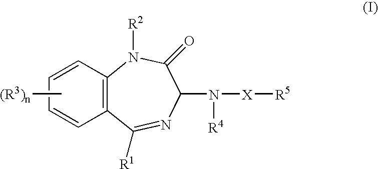

wherein:

R¹ represents C₁₋₆ alkyl, aryl or heteroaryl;

R² represents hydrogen or C₁₋₆ alkyl;

each R³ is the same or different and represents halogen, hydroxy, C₁₋₆ alkyl, C₁₋₆ alkoxy, C₁₋₆ alkylthio, C₁₋₆ haloalkyl, C₁₋₆ haloalkoxy, amino, mono(C₁₋₁₆ alkyl)amino, di(C₁₋₆ alkyl)amino, nitro, cyano, —CO₂R′, —CONR′R″, —NH—CO—R′, —S(O)R′, —S(O)₂R′, —NH—S(O)₂R′, —S(O)NR′R″ or —S(O)₂NR′R″, wherein each R′ and R″ is the same or different and represents hydrogen or C₁₋₆ alkyl;

n is from 0 to 3;

R⁴ represents hydrogen or C₁₋₆ alkyl;

X represents —CO—, —CO—NR′—, —S(O)— or —S(O)₂—, wherein R′ is hydrogen or a C₁-C₆ alkyl group; and

R⁵ represents an aryl, heteroaryl or heterocyclyl group, which group is substituted by a C₁-C₆ hydroxyalkyl group or a —(C₁-C₄ alkyl)-X—(C₁-C₄ alkyl)-X₂—(C₁-C₄ alkyl) group, wherein X₁ represents —O—, —S— or —NR′—, wherein R′ represents H or a C₁-C₄ alkyl group, and X₂ represents —CO—, —SO— or —SO₂—, or R₅ represents -A₁-Y-A₂, wherein:

A₁ is an aryl, heteroaryl, carbocyclyl or heterocyclyl group;

Y represents a direct bond or a C₁-C₄ alkylene, —SO₂—, —CO—, —O—, —S— or —NR′— moiety, wherein R′ is a C₁-C₆ alkyl group; and

A₂ is an aryl, heteroaryl, carbocyclyl or heterocyclyl group.

As used herein, a C₁₋₆ alkyl group or moiety is a linear or branched alkyl group or moiety containing from 1 to 6 carbon atoms, such as a C₁₋₄ alkyl group or moiety. Examples of C₁₋₄ alkyl groups and moieties include methyl, ethyl, n-propyl, i-propyl, n-butyl, i-butyl and t-butyl. For the avoidance of doubt, where two alkyl moieties are present in a group, the alkyl moieties may be the same or different.

As used herein, a hydroxyalkyl group is typically a said alkyl group that is substituted by one or more hydroxy groups. Typically, it is substituted by one, two or three hydroxy groups. Preferably, it is substituted by a single hydroxy group. A preferred hydroxyalkyl group is —CH₂—OH.

As used herein, an acyl group is a C₂₋₇ acyl group, for example a group —CO—R, wherein R is a said C₁₋₆ alkyl group.

As used herein, an aryl group is typically a C₆₋₁₀ aryl group such as phenyl or naphthyl. Phenyl is preferred. An aryl group may be unsubstituted or substituted at any position. Typically, it carries 0, 1, 2 or 3 substituents.

Suitable substituents on an aryl group include halogen, C₁₋₆ alkyl, C₂₋₇ acyl, hydroxy, C₁₋₆ alkoxy, C₁₋₆ alkylthio, C₁₋₆ haloalkyl, C₁₋₆ haloalkoxy, nitro, cyano, carbamoyl, mono(C₁₋₆ alkyl)carbamoyl, di(C₁₋₆ alkyl)carbamoyl, amino, mono(C₁₋₆ alkyl)amino, di(C₁₋₆ alkyl)amino, —CO₂R′, —CONR′R″, —S(O)R′, —S(O)₂R′, —S(O)NR′R″, —S(O)₂NR′R″—NH—S(O)₂R′ or —NH—CO—R′, wherein each R′ and R″ is the same or different and represents hydrogen or C₁₋₆ alkyl.

Preferred substituents on an aryl group include halogen, C₁₋₆ alkyl, C₂₋₇ acyl, hydroxy, C₁₋₆ alkoxy, C₁₋₆ alkylthio, C₁₋₆ haloalkyl, C₁₋₆ haloalkoxy, amino, mono(C₁₋₆ alkyl)amino, di(C₁₋₆ alkyl)amino, nitro, cyano, —CO₂R′, —S(O)R′, —S(O)₂R′ and —S(O)₂NR′R″, wherein each R′ and R″ is the same or different and represents hydrogen or C₁₋₄ alkyl.

Particularly preferred substituents include fluorine, chlorine, bromine, iodine, cyano, C₁₋₄ alkyl, C₂₋₄ acyl, hydroxy, C₁₋₄ alkoxy, C₁₋₄ alkylthio, C₁₋₄ haloalkyl, C₁₋₄ haloalkoxy, amino, mono(C₁₋₄ alkyl)amino, di(C₁₋₄ alkyl)amino, nitro, —CO₂R′, —S(O)₂R′ and —S(O)₂NH₂, wherein R′ represents C₁₋₂ alkyl. Most preferred substituents are chlorine, fluorine, cyano, C₁-C₄ alkyl and C₁-C₄ haloalkyl substituents.

As used herein, references to an aryl group include fused ring systems in which an aryl group is fused to a monocyclic carbocyclyl, heterocyclyl or heteroaryl group or to a fused group which is a monocyclic carbocyclyl, heterocyclyl or heteroaryl group which is fused to a phenyl ring. Typically, said fused ring systems are systems in which an aryl group is fused to a monocyclic carbocyclyl, heterocyclyl or heteroaryl group.

Preferred such fused ring systems are those wherein an aryl group is fused to a monocyclic heterocyclyl or heteroaryl group or to a monocyclic carbocyclic group fused to a phenyl ring, in particular those wherein an aryl group is fused to a heterocyclyl or heteroaryl group. Examples of such fused ring systems are groups in which a phenyl ring is fused to a thienyl group or to a tetrahydrofuranyl group to form a benzothienyl or dihydrobenzofuranyl group. Further examples of such fused rings are groups in which a phenyl ring is fused to a dioxanyl group, a pyrrolyl group or a 2,3-dihydroinden-1-one group to form a benzodioxinyl, indolyl or a 9H-fluoren-9-one group. Most preferably, however, an aryl group, as used herein, is not fused to a monocyclic carbocyclyl, heterocyclyl or heteroaryl group or to a said fused group.

As used herein, a carbocyclyl group is a non-aromatic saturated or unsaturated monocyclic hydrocarbon ring, typically having from 3 to 6 carbon atoms. Preferably it is a saturated hydrocarbon ring (i.e. a cycloalkyl group) having from 3 to 6 carbon atoms. Examples include cyclopropyl, cyclobutyl, cyclopentyl and cyclohexyl. It is preferably cyclopropyl, cyclopentyl or cyclohexyl, most preferably cyclopropyl. A cycloalkyl group may be unsubstituted or substituted at any position. Typically, it carries 0, 1, 2 or 3 substituents.

Suitable substituents on a carbocyclyl group include halogen, C₁₋₆ alkyl, C₂₋₇ acyl, hydroxy, C₁₋₆ alkoxy, C₁₋₆ alkylthio, C₁₋₆ haloalkyl, C₁₋₆ haloalkoxy, nitro, cyano, carbamoyl, mono(C₁₋₆ alkyl)carbamoyl, di(C₁₋₆ alkyl)carbamoyl, amino, mono(C₁₋₆ alkyl)amino, di(C₁₋₆ alkyl)amino, oxo, —CO₂R′, —CONR′R″, —S(O)R′, —S(O)₂R′, —S(O)NR′R″, —S(O)₂NR′R″, —NH—S(O)₂R′ or —NH—CO—R′, wherein each R′ and R″ is the same or different and represents hydrogen or C₁₋₆ alkyl.

Preferred substituents on an carbocyclyl group include halogen, C₁₋₆ alkyl, C₁₋₆ alkoxy, C₁₋₆ alkylthio, C₁₋₆ haloalkyl, C₁₋₆ haloalkoxy, mono(C₁₋₆ alkyl)amino, di(C₁₋₆ alkyl)amino, nitro, cyano and oxo. Particularly preferred substituents include fluorine, chlorine, bromine, C₁₋₄ alkyl, C₁₋₄ alkoxy, C₄ haloalkyl, nitro and oxo. Most preferably, a carbocyclyl group is unsubstituted.

As used herein, a heterocyclyl group is a non-aromatic saturated or unsaturated carbocyclic ring, typically having from 5 to 10 carbon atoms, in which one or more, for example 1, 2 or 3, of the carbon atoms is replaced by a heteroatom selected from N, O and S. Saturated heterocyclyl groups are preferred. Examples include tetrahydrofuranyl, tetrahydrothienyl, pyrrolidinyl, imidazolidinyl, pyrazolidinyl, dioxolanyl, thiazolidinyl, tetrahydropyranyl, piperidinyl, dioxanyl, piperazinyl, morpholinyl, thiomorpholinyl and thioxanyl. Further examples include dithiolanyl, oxazolidinyl, tetrahydrothiopyranyl and dithianyl. Piperazinyl, piperidinyl, thiomorpholinyl, imidazolidinyl and morpholinyl groups are preferred.

As used herein, references to a heterocyclyl group include fused ring systems in which a heterocyclyl group is fused to a phenyl group. Preferred such fused ring systems are those wherein a 5- to 6-membered heterocyclyl group is fused to a phenyl group. An example of such a fused ring system is a group wherein a 1H-imidazol-2(3H)-onyl group or a imidazolidin-2-onyl group is fused to a phenyl ring or a pyridine ring, to form, for example, a 1H-benzo[d]imidazol-2(3H)-onyl group or a 1H-imidazo[4,5-b]pyridin-2(3H)-one group. Most preferably, however, a heterocyclyl group is monocyclic.

A heterocyclic group may be unsubstituted or substituted at any position. Typically, it carries 0, 1 or 2 substituents.

Suitable substituents on a heterocyclyl group include halogen, C₁₋₆ alkyl, C₂₋₇ acyl, hydroxy, C₁₋₆ alkoxy, C₁₋₆ alkylthio, C₁₋₆ haloalkyl, C₁₋₆ haloalkoxy, nitro, cyano, carbamoyl, mono(C₁₋₆ alkyl)carbamoyl, di(C₁₋₆ alkyl)carbamyl, amino, mono(C₁₋₆ alkyl)amino, di(C₁₋₆ alkyl)amino, oxo, —CO₂R′, —CONR′R″, —S(O)R′, —S(O)₂R′, —S(O)NR′R″, —S(O)₂NR′R″, —NH—S(O)₂R′ or —NH—CO—R′, wherein each R′ and R″ is the same or different and represents hydrogen or C₁₋₆ alkyl.

Preferred substituents on a heterocyclyl group include halogen, C₁₋₆ alkyl, C₁₋₆ alkoxy, C₁₋₆ alkylthio, C₁₋₆ haloalkyl, C₁₋₆ haloalkoxy, mono(C₁₋₆ alkyl)amino, di(C₁₋₆ alkyl)amino, nitro, cyano and oxo. Particularly preferred substituents include fluorine, chlorine, bromine, C₁₋₄ alkyl, C₁₋₄ alkoxy, C₁₋₄ haloalkyl, nitro and oxo. Most preferably, a heterocyclyl group is unsubstituted or substituted by one or two C₁₋₂ alkyl or oxo groups. An example of a substituted heterocyclic group is S,S-dioxo-thiomorpholino.

As used herein, a halogen is typically chlorine, fluorine, bromine or iodine. It is preferably chlorine, fluorine or bromine. It is more preferably chlorine or fluorine.

As used herein, an alkoxy group is typically a said alkyl group attached to an oxygen atom. An alkylthio group is typically a said alkyl group attached to a thio group. A haloalkyl or haloalkoxy group is typically a said alkyl or alkoxy group substituted by one or more said halogen atoms. Typically, it is substituted by 1, 2 or 3 said halogen atoms. Preferred haloalkyl and haloalkoxy groups include perhaloalkyl and perhaloalkoxy groups such as —CX₃ and —OCX₃ wherein X is a said halogen atom, for example chlorine or fluorine. Particularly preferred haloalkyl groups are —CF₃ and —CCl₃. Particularly preferred haloalkoxy groups are —OCF₃ and —OCCl₃.

As used herein, a heteroaryl group is typically a 5- to 10-membered aromatic ring, such as a 5- or 6-membered ring, containing at least one heteroatom, for example 1, 2 or 3 heteroatoms, selected from O, S and N. Examples include pyridyl, pyrazinyl, pyrimidinyl, pyridazinyl, furanyl, thienyl, pyrazolidinyl, pyrrolyl, oxadiazolyl, isoxazolyl, thiadiazolyl, thiazolyl, imidazolyl and pyrazolyl groups. Further examples include oxazolyl and isothiazolyl. Preferred heteroaryl groups are pyridyl, thienyl, oxazolyl, isoxazolyl, thiazolyl, furanyl and pyrazolyl.

As used herein, references to a heteroaryl group include fused ring systems in which a heteroaryl group is fused to a phenyl group or to a monocyclic heterocyclyl group. Preferred such fused ring systems are those wherein a 5- to 6-membered heteroaryl group is fused to a phenyl group or to a 5- to 6-membered heterocyclyl group. Examples of such fused ring systems are benzofuranyl, benzothiophenyl, indolyl, benzimidazolyl, benzoxazolyl, quinolinyl, quinazolinyl, isoquinolinyl and 1H-imidazo[4,5-b]pyridin-2(3H)-one moieties. Most preferably, said fused ring system is a 1H-imidazo[4,5-b]pyridin-2(3H)-one moiety.

A heteroaryl group may be unsubstituted or substituted at any position. Typically, it carries 0, 1, 2 or 3 substituents.

Suitable substituents on a heteroaryl group include halogen, C₁₋₆ alkyl, C₂₋₇ acyl, hydroxy, C₁₋₆ alkoxy, C₁₋₆ alkylthio, C₁₋₆ haloalkyl, C₁₋₆ haloalkoxy, nitro, cyano, carbamoyl, mono(C₁₋₆ alkyl)carbamoyl, di(C₁₋₆ alkyl)carbamoyl, amino, mono(C₁₋₆ alkyl)amino, di(C₁₋₆ alkyl)amino, —CO₂R′, —CONR′R″, —S(O)R′, —S(O)₂R′, —S(O)NR′R″, —S(O)₂NR′R″, —NH—S(O)₂R′ or —NH—CO—R′, wherein each R′ and R″ is the same or different and represents hydrogen or C₁₋₆ alkyl.

Preferred substituents on a heteroaryl group include halogen, C₁₋₆ alkyl, C₁₋₆ alkoxy, C₁₋₆ alkylthio, C₁₋₆ haloalkyl, C₁₋₆ haloalkoxy, mono(C₁₋₆ alkyl)amino, di(C₁₋₆ alkyl)amino, nitro and cyano. Particularly preferred substituents include fluorine, chlorine, bromine, C₁₋₄ alkyl, C₁₋₄ alkoxy, C₁₋₄ haloalkyl and nitro. Most preferred substituents include fluorine, chlorine, bromine, C₁₋₂ alkyl and C₁₋₂ haloalkyl substituents.

When R¹ is an aryl or heteroaryl group it is typically unsubstituted or substituted by one, two or three substituents selected from halogen, C₁₋₆ alkyl, C₁₋₆ alkoxy, C₁₋₆ alkylthio, C₁₋₆ haloalkyl or C₁₋₆ haloalkoxy. Preferably, it is unsubstituted or substituted by one or two substituents selected from fluorine, chlorine, bromine, C₁₋₄ alkyl, C₁₋₄ alkoxy, C₁₋₄ alkylthio, C₁₋₄ haloalkyl or C₁₋₄ haloalkoxy. More preferably, it is unsubstituted or substituted by a single fluorine, chlorine, C₁₋₂ alkyl, C₁₋₂ alkoxy, C₁₋₂ alkylthio, C₁₋₂ haloalkyl or C₁₋₂ haloalkoxy substituent.

Typically, R¹ is C₁₋₆ alkyl or aryl. Preferably, R¹ is C₁₋₂ alkyl or aryl. More preferably, R¹ is C₁₋₂ alkyl or phenyl. More preferably, R¹ is an unsubstituted phenyl group.

Typically, R² is hydrogen or C₁₋₄ alkyl. Preferably, R² is hydrogen.

Typically, R³ is halogen, hydroxy, C₁₋₄ alkyl, C₁₋₄ alkoxy, C₁₋₄ alkylthio, C₁₋₄ haloalkyl, C₁₋₄ haloalkoxy, amino, mono(C₁₋₄ alkyl)amino or di(C₁₋₄ alkyl)amino. Preferably, R³ is fluorine, chlorine, bromine, C₁₋₂ alkyl, C₁₋₂ alkoxy, C₁₋₂ alkylthio, C₁₋₂ haloalkyl, C₁₋₂ haloalkoxy, amino, mono(C₁₋₂ alkyl)amino or di(C₁₋₂ alkyl)amino. More preferably, R³ is methyl, trifluoromethyl, fluorine, chlorine or bromine. Most preferably, R³ is methyl or chlorine.

Typically, n is 0, 1 or 2. Preferably, n is 0 or 1. Most preferably, n is 0.

Typically, R⁴ is hydrogen or C₁₋₄ alkyl. Preferably, R⁴ is hydrogen or C₁₋₂ alkyl. More preferably, R⁴ is hydrogen or methyl. Most preferably, R⁴ is hydrogen

Typically, X is —CO—, —S(O)₂— or —CO—NR′—, wherein R′ represents hydrogen or a C₁-C₂ alkyl group. Preferably, X is —CO— or —CO—NR′—.

When R⁵ is a heterocyclyl or heterocyclyl group which is substituted by a C₁-C₆ hydroxyalkyl group or a —(C₁-C₄ alkyl)-X₁—(C₁-C₄ alkyl)-X₂—(C₁-C₄ alkyl) group, the heterocyclyl or heteroaryl group is typically a 5- or 6-membered ring. Preferably, it is a 5- or 6-membered heteroaryl group, for example a furanyl group.

Typically, the C₁-C₆ hydroxyalkyl group is a —CH₂—OH group. Typically, X₁ is —NR′—, wherein R′ is hydrogen or C₁-C₂ alkyl. Typically, X₂ is —S(O)₂—.

Typically, A₁ is an aryl or heteroaryl group. Preferably, A₁ is a monocyclic aryl or heteroaryl group, a naphthyl group or a heteroaryl group fused to a monocyclic oxo substituted heterocyclyl group. More preferably, A₁ is a phenyl group, a monocyclic 5- or 6-membered heteroaryl group or a 5- to 6-membered heteroaryl group fused to a monocyclic oxo substituted 5- to 6-membered heterocyclyl group (for example an oxo substituted imidazolidine group). Most preferably, A₁ is a phenyl, pyridyl, furanyl, thiazolyl, oxazolyl, isoxazolyl, thienyl or 1H-imidazo[4,5-b]pyridin-2-(3H)-one moiety.

Typically, the moiety A₁ is unsubstituted or substituted by 1 or 2 substituents selected from halogen, cyano, nitro, C₁-C₄ alkyl, C₁-C₄ haloalkyl and C₁-C₄ alkoxy substituents. Preferably, the substituents are selected from halogen, cyano, C₁-C₂ alkyl, C₁-C₂ haloalkyl and C₁-C₂ alkoxy substituents.

Typically, Y represents a direct bond, a C₁-C₂ alkylene group, —SO₂— or —O—.

Typically, A₂ is a phenyl, 5- to 6-membered heteroaryl, 5- to 6-membered heterocyclyl or C₃-C₆ cycloalkyl group. Preferably, A₂ is a piperazinyl, pyridyl, morpholinyl, thiomorpholinyl, pyrrolidinyl, piperidinyl, pyrazinyl, cyclopropyl or phenyl group.

Typically, when A₂ is a heterocyclyl group it is attached to the moiety Y via a N atom.

Typically, the moiety A₂ is unsubstituted or substituted by one or two substituents which are selected from C₁-C₄ alkyl and halogen substituents when A₂ is a heteroaryl or aryl group and which are selected from C₁-C₄ alkyl, halogen and oxo substituents when A₂ is a carbocyclic or heterocyclyl group.

Most preferably, A₂ is a piperazinyl, pyridyl, morpholinyl, pyrrolidinyl, piperidinyl, pyrazinyl, cyclopropyl, phenyl or S,S-dioxo-thiomorpholino group, which group is unsubstituted or is substituted by a C₁-C₂ alkyl group.

Preferred compounds of the invention are those in which:

R¹ is C₁₋₆ alkyl or aryl;

R² is hydrogen or C₁₋₄ alkyl;

R³ is halogen, hydroxy, C₁₋₄ alkyl, C₁₋₄ alkoxy, C₁₋₄ alkylthio, C₁₋₄ haloalkyl, C₁₋₄ haloalkoxy, amino, mono(C₁₋₄ alkyl)amino or di(C₁₋₄ alkyl)amino or, preferably, R³ is fluorine, chlorine, bromine, C₁₋₂ alkyl, C₁₋₂ alkoxy, C₁₋₂ alkylthio, C₁₋₂ haloalkyl, C₁₋₂ haloalkoxy, amino, mono(C₁₋₂ alkyl)amino or di(C₁₋₂ alkyl)amino;

n is 0, 1 or 2;

R⁴ is hydrogen or C₁₋₄ alkyl;

X is —CO—, —CO—NR′ or —S(O)₂—, wherein R′ is hydrogen or a C₁-C₂ alkyl group; and

R⁵ is a 5- or 6-membered heterocyclyl or heteroaryl ring which is substituted by a C₁-C₆ hydroxyalkyl group or a —(C₁-C₄ alkyl)-X₁—(C₁-C₄ alkyl)-X₂—(C₁-C₄ alkyl) group, wherein X₁ and X₂ are as defined above, or R⁵ represents -A₁-Y-A₂, wherein:

A₁ is an aryl or heteroaryl group;

Y is a direct bond, a C₁-C₂ alkylene group, —SO₂— or —O—; and

A₂ is an aryl, heteroaryl, heterocyclyl or carbocyclyl group, the aryl moiety in the R¹ group being unsubstituted or substituted by 1, 2 or 3 substituents selected from halogen, C₁-C₆ alkyl, C₁-C₆ alkoxy, C₁-C₆ alkylthio, C₁-C₆ haloalkyl and C₁-C₆ haloalkoxy groups,

the A₁ moiety being unsubstituted or substituted by 1 or 2 substituents selected from halogen, cyano, nitro, C₁-C₄ alkyl, C₁-C₄ haloalkyl and C₁-C₄ alkoxy substituents; and

the A₂ moiety being unsubstituted or substituted by one or two substituents which are selected from C₁-C₄ alkyl and halogen substituents when A₂ is a heteroaryl or aryl group and which are selected from C₁-C₄ alkyl, halogen and oxo substituents when A₂ is a carbocyclic or heterocyclyl group.

Further preferred compounds of the invention are those wherein:

R¹ is C₁₋₂ alkyl or phenyl;

R² is hydrogen or C₁₋₄ alkyl;

R³ is methyl, trifluoromethyl, fluorine, chlorine or bromine;

n is 0 or 1;

R⁴ is hydrogen or C₁₋₂ alkyl;

X is —CO—, —CO—NR′— or —S(O)₂, wherein R′ is hydrogen or a C₁-C₂ alkyl group; and

R⁵ is a 5- or 6-membered heterocyclyl or heteroaryl group which is substituted by a C₁-C₆ hydroxyalkyl group or a —(C₁-C₄ alkyl)-NR′—(C₁-C₄ alkyl)-SO₂—(C₁-C₄ alkyl) group, wherein R′ is hydrogen or C₁-C₂ alkyl, or R⁵ represents -A₁-Y-A₂, wherein:

A₁ is a phenyl group, a monocyclic 5- or 6-membered heteroaryl group or a 5- or 6-membered heteroaryl group fused to a monocyclic oxo-substituted 5- to 6-membered heterocyclyl group;

Y represents a direct bond, a C₁-C₂ alkylene moiety, —SO₂— or —O—; and

A₂ is a phenyl, 5- to 6-membered heteroaryl, 5- to 6-membered heterocyclyl or C₃-C₆ cycloalkyl group,

the phenyl moiety in the R¹ group being unsubstituted or substituted by one or two substituents selected from fluorine, chlorine, bromine, C₁₋₄ alkyl, C₁₋₄ alkoxy, C₁₋₄ alkylthio, C₁₋₄ haloalkyl or C₁₋₄ haloalkoxy;

the A₁ moiety being unsubstituted or substituted by 1 or 2 substituents selected from halogen, cyano, nitro, C₁-C₄ alkyl, C₁-C₄ haloalkyl and C₁-C₄ alkoxy substituents; and

the A₂ moiety being unsubstituted or substituted by 1 or 2 substituents which are selected from C₁-C₄ alkyl, halogen and oxo substituents when A₂ is a heterocyclyl or cycloalkyl group and which are selected from C₁-C₄ alkyl and halogen substituents when A₂ is a phenyl or heteroaryl group.

Particularly preferred compounds of the invention are compounds of formula (Ia) and pharmaceutically acceptable salts thereof

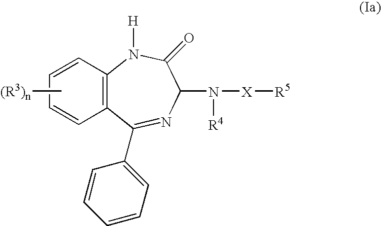

wherein:

X is —CO— or —CO—NH—; and

R⁵ is a 5- to 6-membered heteroaryl group, for example a furanyl group, which is substituted by —CH₂—OH or —(C₁-C₄ alkyl)-N(CH₃)—(C₁-C₄ alkyl)-SO₂—(C₁-C₄ alkyl) or R₅ represents -A₁-Y-A₂, wherein:

A₁ is a phenyl, pyridyl, furanyl, thiazolyl, oxazolyl, isoxazolyl, thienyl or 1H-imidazo[4,5-b]pyridin-2-(3H)-one moiety, which is unsubstituted or substituted by 1 or 2 substituents selected from halogen, cyano, C₁-C₂ alkyl, C₁-C₂ haloalkyl and C₁-C₂ alkoxy substituents;

Y is a direct bond, a C₁-C₂ alkylene group, —SO₂— or —O—; and

A₂ is a piperazinyl, pyridyl, morpholinyl, pyrrolidinyl, piperidinyl, pyrazinyl, cyclopropyl, phenyl or S,S-dioxo-thiomorpholino group, which is unsubstituted or substituted by a C₁-C₂ alkyl group.

In the compounds of formula (Ia), typically n is 0 and R₄ is hydrogen.

Compounds of the formula (I) containing one or more chiral centre may be used in enantiomerically or diasteroisomerically pure form, or in the form of a mixture of isomers. For the avoidance of doubt, the chemical structures depicted herein are intended to embrace all stereoisomers of the compounds shown, including racemic and non-racemic mixtures and pure enantiomers and/or diastereoisomers.

Preferred compounds of the invention are optically active isomers. Thus, for example, preferred compounds of formula (I) containing only one chiral centre include an R enantiomer in substantially pure form, an S enantiomer in substantially pure form and enantiomeric mixtures which contain an excess of the R enantiomer or an excess of the S enantiomer. For the avoidance of doubt, the compounds of the formula (I) can, if desired, be used in the form of solvates.

As used herein, a pharmaceutically acceptable salt is a salt with a pharmaceutically acceptable acid or base. Pharmaceutically acceptable acids include both inorganic acids such as hydrochloric, sulphuric, phosphoric, diphosphoric, hydrobromic or nitric acid and organic acids such as citric, fumaric, maleic, malic, ascorbic, succinic, tartaric, benzoic, acetic, methanesulphonic, ethanesulphonic, benzenesulphonic or p-toluenesulphonic acid. Pharmaceutical acceptable bases include alkali metal (e.g. sodium or potassium) and alkaline earth metal (e.g. calcium or magnesium) hydroxides and organic bases such as alkyl amines, aralkyl amines or heterocyclic amines.

Particularly preferred compounds of the invention include:
6-(4-Methyl-piperazin-1-yl)-N-(2-oxo-5-phenyl-2,3-dihydro-1H-benzo[e][1,4]diazepin-3-yl)-nicotinamide;
3,4,5,6-Tetrahydro-2H-[1,2′]bipyridinyl-5′-carboxylic acid (2-oxo-5-phenyl-2,3-dihydro-1H-benzo[e][1,4]diazepin-3-yl)-amide;
(S)-2-(1,1-Dioxo-1λ6-thiomorpholin-4-yl)-N-(2-oxo-5-phenyl-2,3-dihydro-1H-benzo[e][1,4]diazepin-3-yl-benzamide;
(S)-2-Chloro-4-morpholin-4-yl-N-(2-oxo-5-phenyl-2,3-dihydro-1H-benzo[e][1,4]diazepin-3-yl)-benzamide;
(S)-2-(1,1-Dioxo-1λ6-thiomorpholin-4-yl)-4-fluoro-(2-oxo-5-phenyl-2,3-dihydro-1H-benzo[e][1,4]diazepin-3-yl-benzamide;
(S)-5-Chloro-2-(1,1-dioxo-1λ6-thiomorpholin-4-yl)-N-(2-oxo-5-phenyl-2,3-dihydro-1H-benzo[e][1,4]diazepin-3-yl)-benzamide;
(S)-2-(1,1-Dioxo-1λ6-thiomorpholin-4-yl)-5-fluoro-N-(2-oxo-5-phenyl-2,3-dihydro-1H-benzo[e][1,4]diazepin-3-yl)-benzamide;
(S)-5-(4-Methyl-piperazin-1-ylmethyl)-furan-2-carboxylic acid (2-oxo-5-phenyl-2,3-dihydro-1H-benzo[e][1,4]diazepin-3-yl)-amide;
(S)-5-Pyrrolidin-1-ylmethyl-furan-2-carboxylic acid (2-oxo-5-phenyl-2,3-dihydro-1H-benzo[e][1,4]diazepin-3-yl)-amide;
(S)-5-Piperidin-1-ylmethyl-furan-2-carboxylic acid (2-oxo-5-phenyl-2,3-dihydro-1H-benzo[e][1,4]diazepin-3-yl)-amide;

(S)-5-Dimethylaminomethyl-furan-2-carboxylic acid (2-oxo-5-phenyl-2,3-dihydro-1H-benzo[e][1,4]diazepin-3-yl)-amide;
(S)-4-Fluoro-N-(2-oxo-5-phenyl-2,3-dihydro-1H-benzo[e][1,4]diazepin-3-yl)-2-piperidin-1-yl-benzamide;
(S)-4-Fluoro-2-morpholino-4-yl-N-(2-oxo-5-phenyl-2,3-dihydro-1H-benzo[e][1,4]diazepin-3-yl)-benzamide;
(S)-4-Cyano-N-(2-oxo-5-phenyl-2,3-dihydro-1H-benzo[e][1,4]diazepin-3-yl)-2-pyrrolidin-1-yl-benzamide;
(S)-4-Cyano-N-(2-oxo-5-phenyl-2,3-dihydro-1H-benzo[e][1,4]diazepin-3-yl)-piperidine-1-yl-benzamide;

(S)—N-(2-Oxo-5-phenyl-2,3-dihydro-1H-benzo[e][1,4]diazepin-3-yl)-2-pyrrolidin-1-yl-4-trifluoromethyl-benzamide;

(S)—N-(2-Oxo-5-phenyl-2,3-dihydro-1H-benzo[e][1,4]diazepin-3-yl)-2-piperidin-1-yl-4-trifluoromethyl-benzamide;
(S)-2-Morpholin-4-yl-N-(2-oxo-5-phenyl-2,3-dihydro-1H-benzo[e][1,4]diazepin-3-yl)-4-trifluoromethyl-benzamide;

(S)—N-(2-Oxo-5-phenyl-2,3-dihydro-1H-benzo[e][1,4]diazepin-3-yl)-2-pyrrolidin-1-yl-5-trifluoromethyl-benzamide;
(S)-2-Morpholin-4-yl-N-(2-oxo-5-phenyl-2,3-dihydro-1H-benzo[e][1,4]diazepin-3-yl)-5-trifluoromethyl-benzamide;
(S)-2-Morpholin-4-yl-N-(2-oxo-5-phenyl-2,3-dihydro-1H-benzo[e][1,4]diazepin-3-yl)-nicotinamide;
(S)-2-(1,1-Dioxo-1λ6-thiomorpholin-4-yl)-N-(2-oxo-5-phenyl-2,3-dihydro-1H-benzo[e][1,4]diazepin-3-yl)-nicotinamide;
(S)-2-(1,1-Dioxo-1λ6-thiomorpholin-4-yl)-3-methyl-N-(2-oxo-5-phenyl-2,3-dihydro-1H-benzo[e][1,4]diazepin-3-yl)-benzamide;
(S)-2-(1,1-Dioxo-1λ6-thiomorpholin-4-yl)-4-methyl-N-(2-oxo-5-phenyl-2,3-dihydro-1H-benzo[e][1,4]diazepin-3-yl)-benzamide;
(S)-2-(1,1-Dioxo-1λ6-thiomorpholin-4-yl)-6-methyl-N-(2-oxo-5-phenyl-2,3-dihydro-1H-benzo[e][1,4]diazepin-3-yl)-benzamide;
(S)-2-Chloro-6-(1,1-dioxo-1λ6-thiomorpholin-4-yl)-N-(2-oxo-5-phenyl-2,3-dihydro-1H-benzo[e][1,4]diazepin-3-yl)-benzamide;
(S)-3-Cyclopropyl-2-oxo-2,3-dihydro-imidazo[4,5-b]pyridine-1-carboxylic acid (2-oxo-5-phenyl-2,3-dihydro-1H-benzo[e][1,4]diazepin-3-yl)-amide;
(S)-3-(4-Methyl-piperazine-1-sulfonyl)-N-(2-oxo-5-phenyl-2,3-dihydro-1H-benzo[e][1,4]diazepin-3-yl)-benzamide;
(S)-4-(4-Methyl-piperazin-1-yl)-N-(2-oxo-5-phenyl-2,3-dihydro-1H-benzo[e][1,4]diazepin-3-yl)-benzamide;
(S)—N-(2-Oxo-5-phenyl-2,3-dihydro-1H-benzo[e][1,4]diazepin-3-yl)-3-(piperidine-1-sulfonyl)-benzamide;
(S)-3-(Morpholine-4-sulfonyl)-N-(2-oxo-5-phenyl-2,3-dihydro-1H-benzo[e][1,4]diazepin-3-yl)-benzamide;
(S)-5-Morpholin-4-ylmethyl-furan-2-carboxylic acid (2-oxo-5-phenyl-2,3-dihydro-1H-benzo[e][1,4]diazepin-3-yl)-amide;
(S)-5-Hydroxymethyl-furan-2-carboxylic acid (2-oxo-5-phenyl-2,3-dihydro-1H-benzo[e][1,4]diazepin-3-yl)-amide;
(S)-5-(1,1-Dioxo-1λ6-thiomorpholin-4-ylmethyl)-furan-2-carboxylic acid (2-oxo-5-phenyl-2,3-dihydro-1H-benzo[e][1,4]diazepin-3-yl)-amide;
(S)-2-Chloro-4-(1,1-dioxo-1λ6-thiomorpholin-4-yl)-N-(2-oxo-5-phenyl-2,3-dihydro-1H-benzo[e][1,4]diazepin-3-yl)-benzamide;
(S)-2-Chloro-5-(1,1-dioxo-1λ6-thiomorpholin-4-yl)-N-(2-oxo-5-phenyl-2,3-dihydro-1H-benzo[e][1,4]diazepin-3-yl)-benzamide;
(S)-5-{[(2-Methanesulfonyl-ethyl)-methyl-amino]-methyl}-furan-2-carboxylic acid

(2-oxo-5-phenyl-2,3-dihydro-1H-benzo[e][1,4]diazepin-3-yl-amide;
(S)-2-Pyridin-3-yl-thiazole-4-carboxylic acid (2-oxo-5-phenyl-2,3-dihydro-1H-benzo[e][1,4]diazepin-3-yl)-amide;
(S)-2-Pyridin-4-yl-thiazole-4-carboxylic acid (2-oxo-5-phenyl-2,3-dihydro-1H-benzo[e][1,4]diazepin-3-yl)-amide;
(S)-4-Methyl-2-pyrazin-2-yl-thiazole-5-carboxylic acid (2-oxo-5-phenyl-2,3-dihydro-1H-benzo[e][1,4]diazepin-3-yl)-amide;
(S)-2-Morpholin-4-ylmethyl-furan-3-carboxylic acid (2-oxo-5-phenyl-2,3-dihydro-1H-benzo[e][1,4]diazepin-3-yl)-amide;
(S)-3-Morpholin-4-ylmethyl-N-(2-oxo-5-phenyl-2,3-dihydro-1H-benzo[e][1,4]diazepin-3-yl)-benzamide;
(S)-5-Morpholin-4-ylmethyl-isoxazole-3-carboxylic acid (2-oxo-5-phenyl-2,3-dihydro-1H-benzo[e][1,4]diazepin-3-yl)-amide;
(S)-3-Morpholin-4-ylmethyl-furan-2-carboxylic acid (2-oxo-5-phenyl-2,3-dihydro-1H-benzo[e][1,4]diazepin-3-yl)-amide;
(S)-5-Pyridin-2-yl-thiophene-2-carboxylic acid (2-oxo-5-phenyl-2,3-dihydro-1H-benzo[e][1,4]diazepin-3-yl)-amide;
(S)-2-Methyl-4-(morpholin-4-sulfonyl)-furan-3-carboxylic acid (2-oxo-5-phenyl-2,3-dihydro-1H-benzo[e][1,4]diazepin-3-yl)-amide;
(S)-6-Morpholin-4-yl-N-(2-oxo-5-phenyl-2,3-dihydro-1H-benzo[e][1,4]diazepin-3-yl)-nicotinamide;
(S)-3-Morpholin-4-ylmethyl-thiophene-2-carboxylic acid (2-oxo-5-phenyl-2,3-dihydro-1H-benzo[e][1,4]diazepin-3-yl)-amide;
(S)-5-Morpholin-4-ylmethyl-thiophene-2-carboxylic acid (2-oxo-5-phenyl-2,3-dihydro-1H-benzo[e][1,4]diazepin-3-yl)-amide;
2-Morpholin-4-yl-N-(2-oxo-5-phenyl-2,3-dihydro-1H-benzo[e][1,4]diazepin-3-yl)-benzamide;
(S)-5-Phenyl-oxazole-4-carboxylic acid (2-oxo-5-phenyl-2,3-dihydro-1H-benzo[e][1,4]diazepin-3-yl)-amide;
1-(2-Oxo-5-phenyl-2,3-dihydro-1H-benzo[e][1,4]diazepin-3-yl)-3-(4-phenoxy-phenyl)-urea;
   an N-oxide of any of the above compounds;
   and pharmaceutically acceptable salts thereof.

Compounds of formula (I) may be prepared by reacting glyoxylic acid (HCO—CO₂H), benzotriazole and an appropriate benzyl carbamate at reflux in toluene, under Dean-Stark conditions giving the key protected amino acid of formula (II)

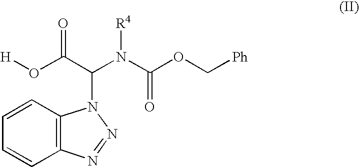

The thus obtained amino acid of formula (II) can then be reacted with a suitable chlorinating agent, such as oxalyl chloride, followed by reaction with a 2-aminobenzophenone of formula (III)

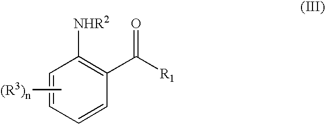

to give the intermediate amide of formula (IV)

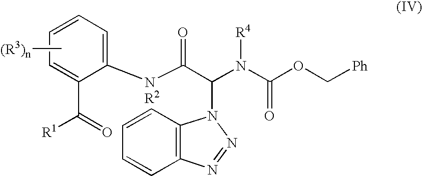

which need not be characterized.

The compound of formula (IV) can then be subjected to ammonolysis followed by ring closure in acetic acid containing ammonium acetate to obtain the protected benzodiazepine of formula (V)

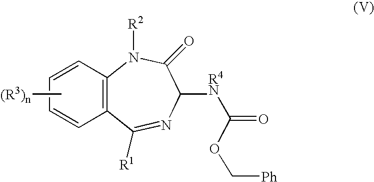

The compound of formula (V) can then be deprotected using hydrogen bromide in acetic acid to yield the deprotected amine of formula (VI).

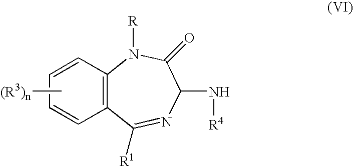

Compounds of formula (I), in which X is —CO— or —CO—NR′ can be prepared by reacting a compound of formula (VI), as defined above, with an acid anhydride in a suitable solvent, preferably pyridine at ambient temperature, or with an acid chloride in a suitable solvent in the presence of a base, preferably in THF at ambient temperature with triethylamine present. Alternatively, the compounds can be produced by reaction of a compound of formula (VI) with an acid in a suitable solvent in the presence of a base and a coupling agent, preferably in THF at ambient temperature with triethylamine and O-benzotriazol-1-yl-N,N, N′,N′-tetramethyluronium hexafluorophosphate (HBTU) present.

If the acid chloride used is an amino carbonyl chloride, the compound of formula (I) is a urea. In the case where R′ in the X moiety is hydrogen, such compounds may also be prepared by the reaction of a compound of formula (VI) with an isocyanate. This reaction is preferably carried out in THF at ambient temperature. Alternatively, the isocyanate may be prepared in situ from the relevant amine and phosgene, in the presence of a base, usually triethylamine, again in THF. Compounds in which R′ is other than hydrogen can, of course, be prepared by reacting a corresponding compound in which R′ is hydrogen with an appropriate alkylating agent, for example L-(C₁-C₆ alkyl) wherein L is a leaving group, for example chlorine.

Compounds of formula (I), in which X is —S(O)₂— may be prepared by the reaction of a compound of formula (VI) with a suitable sulfonyl chloride. Similarly, compounds of formula (I), in which X is —S(O)— may be prepared by the reaction of a compound of formula (VI) with a suitable sulfinyl chloride

In the preparation of the benzodiazepine skeleton, commercially available aminobenzophenone compounds of formula (III) can be used where possible. Compounds of formula (III) which are not commercially available can be prepared by known methods, for example by reaction of a Weinreb type amide of formula (VII)

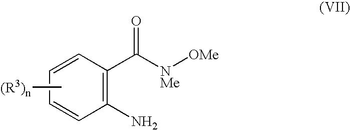

with a group R¹—Li or a Grignard reagent such as R¹—MgBr. Preferably this reaction is carried out in THF at −100° C.

Compounds of formula (VII) are known compounds or can be prepared by analogy with known methods. For example, they can be prepared from the reaction of isatoic anhydrides of formula (VIII)

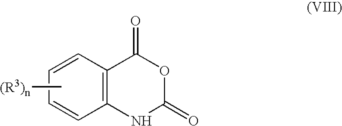

with N,O-dimethyl hydroxylamine under standard reaction conditions.

The starting materials of formula (II), (III), (VII), and (VIII) are known compounds, or may be prepared by analogy with known methods.

Further synthetic manipulation of the thus obtained compounds of formula (I) may be carried out by conventional methods to achieve further compounds of formula (I). The benzodiazepines of formula (I) can be salified by treatment with an appropriate acid or base.

Although the described route to the claimed compounds provides an adequate synthesis for laboratory scale preparations, an alternative route was sought which has potential as a manufacturing route. The same starting material (2-amino-benzophenone) (1) is used in both, however in the alternative route, the benzodiazepine ring system is formed by reaction initially with bromoacetyl bromide (or an equivalent reagent) followed by ring closure with ammonia. These reactions are carried out in a suitable solvent, such as dichloromethane, and at a suitable temperature which may range from −20 to 150° C. In order to protect the NH functionality, at this stage the unsubstituted benzodiazepine is reacted with a base, and an alkylating agent. For instance sodium hydride in DMF followed by addition of 4-methoxy-benzyl chloride gives rise to the intermediate (2) shown below. Further reaction of this material with a base (e.g. potassium tert-butoxide) in a suitable solvent (e.g. THF or DMF) followed by quenching with isoamyl nitrite (or an alternative similar reagent) furnishes the oxime intermediate (3) which may be converted into the racemic primary amine by methods which include the use of hydrogen and a suitable catalyst. This amine then undergoes a Dynamic Kinetic Resolution (DKR) procedure by which the racemic amine in the presence of a suitable optically active acid, and a suitable aldehyde gives rise to precipitation of the salt of the desired (S)-amine (4) in good yield and exceptionally high enantiomeric excess. A suitable acid for this conversion can be e.g. Camphorsulfonic acid, Boc-phenyl alanine or the like, and a suitable aldehyde may be a benzaldehyde such as 3,5-dichloro salicylaldehyde.

The optically amine thus formed may then be transformed into a desired derivative, such as an amide or urea. The amide formations may be carried out using a suitable carboxylic acid and a coupling reagent, or a carbonyl chloride or other suitable reagent, and the ureas prepared using either a suitable isocyanate, or alternatively reaction with phosgene followed by a suitable amine.

These derivatives thus formed may then have the protecting group removed. This may be carried out in the presence of a Lewis Acid, such as aluminium chloride, boron trifluoride, titanium tetrachloride, or the like. These reactions are carried out in a suitable inert solvent, such as dichloromethane. Reaction temperatures may range from −20 to 150° C., but are typically carried out at room temperature or below.

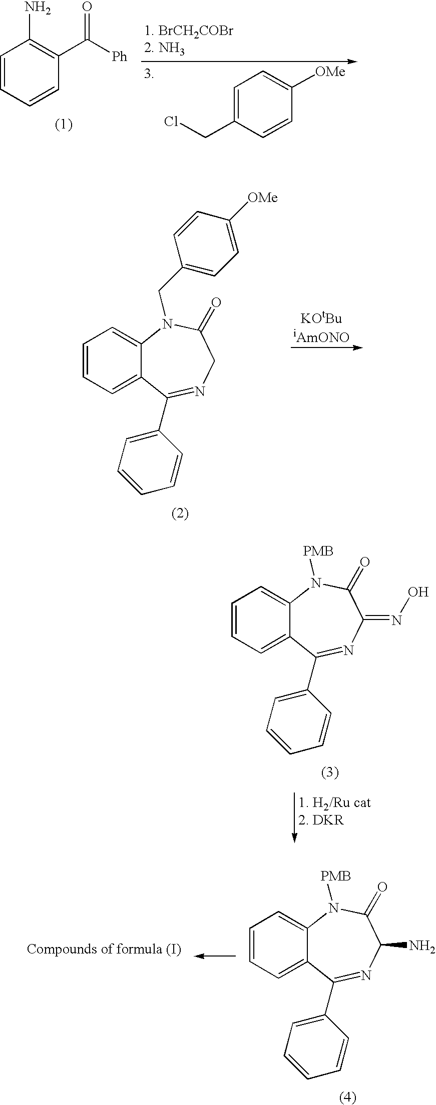

As explained above, the compounds of the invention are active against RSV. The present invention therefore provides a method for treating a patient suffering from or susceptible to an RSV infection, which method comprises administering to said patient an effective amount of a compound of formula (I) or a pharmaceutically acceptable salt thereof.

RSV is prevalent among children younger than two years of age, adults suffering from asthma, chronic obstructive pulmonary disorder (COPD) or immunodeficiency and the elderly. It is a particularly serious risk amongst children who suffer from chronic lung disease. Accordingly, the said composition or medicament is typically for use in treating a patient who is a child under two years of age, patients with asthma, COPD or immunodeficiency the elderly or persons in long term care facilities. Typically, said child suffers from chronic lung disease.

Further, anti-RSV prophylaxis is recommended for infants born at 32 weeks of gestation or earlier, until they reach 6 months of age, the elderly, persons with immunedeficiency and those in long term care facilities. Accordingly, the said composition or medicament is typically for use in preventing RSV infection in an infant less than 6 years of age, who was born after 32 weeks of gestation or less, the elderly, persons with immunosufficiency and those in long term care facilities.

It has been shown that RSV infections are accompanied by inflammatory reactions (Noah et al, Clinical Immunology 2000, Vol 97, 43-49). The present invention also relates to a combination of a compound of formula (I), or a pharmaceutically acceptable salt thereof, with an anti-inflammatory compound and the use of such a combination in the treatment of RSV. Typically, said anti-inflammatory compound is a steroid, for example budesonide or fluticasone, a non-steroid, for example a leukotriene antagonist, phosphodiesterase 4 inhibitor or TNF alpha inhibitor or an interleukin 8 or interleukin 9 inhibitor.

Thus, in one embodiment, a compound of formula (I), or pharmaceutically acceptable salt thereof, is combined with a steroid antiinflammatory compound, for example budesonide or fluticasone. In a preferred embodiment, the steroid is administered in low doses to minimize immuno-suppressant effects. In another embodiment a compound of formula (I), or a pharmaceutically acceptable salt thereof, is combined with a non-steroid anti-inflammatory compound, for example leukotriene antagonists such as Singulair (Merck) or Accolate (Astra Zeneca), phosphodiesterase 4 inhibitors such as roflumilast (Altana), TNF alpha inhibitors such as Enbrel (Amgen), Remicade (Centocor), Humira (Abbott) or CDP870 (Celltech) or NSAIDS. In a further embodiment, a compound of formula (I) is combined with interleukin 8 or interleukin 9 inhibitors. The present invention thus also relates to a product containing a compound of formula (I), or a pharmaceutically acceptable salt thereof, and an anti-inflammatory compound for simultaneous, separate or sequential use in the treatment of RSV.

The present invention also relates to a combination of a compound of formula (I), or a pharmaceutically acceptable salt thereof, with an anti-influenza compound and the use of such a combination in the treatment of concomitant RSV and influenza infections. The present invention thus also relates to a product containing a compound of formula (I), or a pharmaceutically acceptable salt thereof, and an anti-influenza compound for simultaneous, separate or sequential use in the treatment of concomitant RSV and influenza infections.

It is a further surprising finding of the present invention that compounds of the invention are active against human metapneumovirus, measles, parainfluenza viruses, paramyxoviruses and mumps. The present invention thus provides the use of a compound of formula (I), or a pharmaceutically acceptable salt thereof, in the manufacture of a medicament for use in the treatment of human metapneumovirus, measles, parainfluenza viruses, paramyxoviruses and mumps.

The compounds of the invention may be administered in a variety of dosage forms. Thus, they can be administered orally, for example as tablets, troches, lozenges, aqueous or oily suspensions, dispersible powders or granules. The compounds of the invention may also be administered parenterally, whether subcutaneously, intravenously, intramuscularly, intrasternally, transdermally or by infusion techniques. The compounds may also be administered as suppositories.

In a preferred embodiment, the compounds of the invention are administered by intranasal or intrabronchial administration. The present invention also provides an inhaler or nebuliser containing a medicament which comprises (a) a benzodiazepine derivative of the formula (I), as defined above, or a pharmaceutically acceptable salt thereof, and (b) a pharmaceutically acceptable carrier or diluent.

The present invention also provides a pharmaceutical composition containing such a benzodiazepine derivative, or a pharmaceutically acceptable salt thereof, and a pharmaceutically acceptable carrier or diluent.

Said pharmaceutical composition typically contains up to 85 wt % of a compound of the invention. More typically, it contains up to 50 wt % of a compound of the invention. Preferred pharmaceutical compositions are sterile and pyrogen free. Further, the pharmaceutical compositions provided by the invention typically contain a compound of the invention which is a substantially pure optical isomer.

The compounds of the invention are typically formulated for administration with a pharmaceutically acceptable carrier or diluent. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non toxic and pharmacologically inactive substances used in pharmaceutical formulations. Such pharmaceutical preparations may be manufactured in known manner, for example, by means of mixing, granulating, tableting, sugar coating, or film coating processes.

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspension or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for injection or infusion may contain as carrier, for example, sterile water or preferably they may be in the form of sterile, aqueous, isotonic saline solutions.

A therapeutically effective amount of a compound of the invention is administered to a patient. A typical dose is from about 0.001 to 50 mg per kg of body weight, according to the activity of the specific compound, the age, weight and conditions of the subject to be treated, the type and severity of the disease and the frequency and route of administration. Preferably, daily dosage levels are from 5 mg to 2 g.

Certain benzodiazepine derivatives of the formula (I) are novel per se. The present invention includes these novel compounds and pharmaceutically acceptable salts thereof. The present invention therefore also provides compounds of formula (Ib), or a pharmaceutically acceptable salt thereof

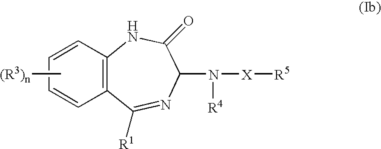

wherein R₁, R₃, n, R₄, X and R₅ are as defined above.

Typically, in the formula (Ib), R₁ is an unsubstituted phenyl group.

Typically, in the formula (Ib), when A₁ is a heteroaryl group, it is other than a 5-methyl-isoxazolyl moiety.

Typically, in the formula (Ib), A₁ is an aryl or heteroaryl moiety.

Typically, in the formula (Ib), X is —CO— or —CO—NR′—, wherein R′ is as defined above, provided that when X is —CO—NR′—, the moiety -A₁-Y-A₂ is -phenyl-O-phenyl.

Typically, in the formula (Ib), A₂ is other than a 4- to 10-membered saturated cycloalkyl ring, in which one of the carbon atoms is replaced by a N atom. In particular, A₂ is typically other than a substituted or unsubstituted moiety of the formula

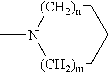

wherein n and m are the same or different and each represent an integer of from 1 to 4.

Typically, in the formula (Ib), A₂ is a piperazinyl, pyridyl, pyrrolidinyl, pyrazinyl, cyclopropyl, phenyl or S,S-dioxo-thiomorpholino group which is unsubstituted or is substituted by a C₁-C₂ alkyl group.

The present invention also relates to the novel compounds, as defined above, or a pharmaceutically acceptable salt thereof, for use in a method of treating the human or animal body. The present invention also relates to a pharmaceutical composition comprising a novel compound as defined above and a pharmaceutically acceptable diluant or carrier. Preferably, the pharmaceutical composition comprises a pharmaceutically acceptable salt of a novel compound as defined above. A pharmaceutically acceptable salt is as defined above. The novel compounds of the invention are typically administered in the manner defined above and the compounds are typically formulated for administration in the manner defined above.

Preferably, the pharmaceutical compositions comprise optically active isomers of the novel compounds of the invention. Thus, for example, preferred novel compounds of the invention containing only one chiral centre include an R enantiomer in substantially pure form, an S enantiomer in substantially pure form and enantiomeric mixtures which contain an excess of the R enantiomer or an excess of the S enantiomer. It is particularly preferred that pharmaceutical contains a compound of the invention which is a substantially pure optical isomer. For the avoidance of doubt, the novel compounds of the invention can, if desired, be used in the form of solvates.

The following Examples illustrate the invention. They do not however, limit the invention in any way. In this regard, it is important to understand that the particular assays used in the Examples section are designed only to provide an indication of anti-RSV activity. There are many assays available to determine the activity of given compounds against RSV, and a negative result in any one particular assay is therefore not determinative.

### EXAMPLES

### Intermediate 1

### 2-Chloro-4-(1,1-dioxo-1λ6-thiomorpholin-4-yl)-benzoic acid

A mixture of 4-amino-2-chlorobenzoic acid (172 mg) and ethenesulfonyl-ethene (0.15 ml) in water (3 ml) containing sodium carbonate (212 mg) was heated to 100 C for 18 h. The mixture was allowed to cool and was acidified with 2N HCl. The off-white precipitate was collected and dried (263 mg)

LC/MS RT=4.09 mins, ES− 288,290

### Intermediate 2

### 2-Chloro-5-(1,1-dioxo-1λ6-thiomorpholin-4-yl)-benzoic acid

A mixture of 5-amino-2-chlorobenzoic acid (172 mg) and ethenesulfonyl-ethene (0.15 ml) in water (3 ml) was heated to 10° C. for 18 h. The mixture was allowed to cool and was extracted with dichloromethane. The dried extracts were evaporated giving a pale brown solid (265 mg)

LC/MS RT=4.13 mins, ES− 288,290

### Intermediate 3

### 2-(1,1-Dioxo-1λ6-thiomorpholin-4-yl)-nicotinic acid

This material was prepared as described for Intermediate 1 except that 2-amino-nicotinic acid (138 mg) was used. The title compound was isolated as an off-white solid (93 mg)

### Intermediate 4

### 2-(1,1-Dioxo-1λ6-thiomorpholin-4-yl)-3-methyl-benzoic acid

This material was prepared as described for Intermediate 2 except that 2-amino-3-methyl-benzoic acid (302 mg) was used. The title compound was isolated as a pale brown solid (486 mg)

### Intermediate 5

### 2-(1,1-Dioxo-1λ6-thiomorpholin-4-yl)-4-methyl-benzoic acid

This material was prepared as described for Intermediate 2 except that 2-amino-4-methyl-benzoic acid (302 mg) was used. The title compound was isolated as a brown solid (430 mg)

### Intermediate 6

### 2-(1,1-Dioxo-1λ6-thiomorpholin-4-yl)-6-methyl-benzoic acid

This material was prepared as described for Intermediate 2 except that 2-amino-6-methyl-benzoic acid (302 mg) was used. The title compound was isolated as a brown solid (490 mg)

### Intermediate 7

### 3-(4-Methyl-piperazine-1-sulfonyl)-benzoic acid

A solution of 3-chlorosulfonyl-benzoic acid (89 mg) 4-dimethylamino-pyridine (catalytic amount) and N-methylpiperazine (0.045 ml) in dichloromethane (10 ml) was heated to reflux for 2 h. The solvent was then evaporated and the crude material used without purification or characterisation in the next synthetic step.

### Intermediate 8

### 3-Piperidine-1-sulfonyl-benzoic acid

This material was prepared as described for Intermediate 7 except that piperidine was used as the nucleophile. As for Intermediate 7 the material was used crude.

### Intermediate 9

### 3-(Morpholine-4-sulfonyl)-benzoic acid

This material was prepared as described for Intermediate 7 except that morpholine was used as the nucleophile. As for Intermediate 7 the material was used crude.

### Intermediate 10

### 2-Chloro-6-(1,1-dioxo-1λ6-thiomorpholin-4-yl)-benzoic acid

This material was prepared as described for Intermediate 2 except that 2-amino-6-chloro-benzoic acid (343 mg) was used. The title compound was isolated as a buff solid (405 mg)

### Intermediate 11

### 5-Chloro-2-(1,1-dioxo-1λ6-thiomorpholin-4-yl)-benzoic acid

This material was prepared as described for Intermediate 2 except that 2-amino-5-chloro-benzoic acid (200 mg) was used. The title compound was isolated as a white solid (233 mg)

¹H NMR (DMSO, δ) 3.25 (brs, 4H) 3.47 (brs, 4H) 7.31 (d, 1H) 7.54 (dd, 1H) 7.71 (d, 1H)

LC/MS RT=4.66 min Found ES⁺=290,292

### Intermediate 12

### 2-(1,1-Dioxo-1λ6-thiomorpholin-4-yl)-5-fluoro-benzoic acid

This material was prepared as described for Intermediate 2 except that 2-amino-5-fluoro-benzoic acid (200 mg) was used. The title compound was isolated as a white solid (310 mg)

¹H NMR (DMSO, δ) 3.28 (m, 4H) 3.42 (m, 4H) 7.33-7.56 (m, 3H)

LC/MS RT=4.28 min Found ES⁻=272

### Intermediate 13

### 4-Fluoro-2-thiomorpholin-4-yl-benzoic acid

A mixture of 2,4-difluoro-benzoic acid (0.5 g), thiomorpholine (0.33 ml) and triethylamine (0.88 ml) in acetonitrile (2 ml) was heated to 200 C in a microwave reactor for 20 mins. The residue was partitioned between water and dichloromethane. The dried organic layer was evaporated and then purified on a silica gel SPE cartridge. Elution with dichloromethane followed by a gradient of dichloromethane:ethanol:0.880 ammonia; 800:8:1 to 200:8:1 gave the title material as a white solid (292 mg)

¹H NMR (DMSO, δ) 2.81 (m, 4H) 3.27 (m, 4H) 7.11 (m, 1H) 7.40 (dd, 1H) 7.95 (m, 1H)

### Intermediate 14

### 2-(1,1-Dioxo-4-oxy-1λ6-thiomorpholin-4-yl)-4-fluoro-benzoic acid

Intermediate 11 (262 mg) and potassium peroxymonosulfate (1.34 g) in methanol (5 ml) and water (2.5 ml) was stirred at room temperature for 6 h. The precipitate formed was collected by filtration then dissolved in aqueous sodium bicarbonate. Acidification to pH3 with 1M HCl led to the formation of a white precipitate which was collected and dried (194 mg)

¹H NMR (DMSO, δ) 3.2-3.48 (brm, 4H) 3.59 (t, 2H) 3.89 (t, 2H) 6.96 (m, 1H) 7.30 (dd, 1H) 7.85 (m, 1H)

### Intermediate 15

### 6-Chloro-N-(2-oxo-5-phenyl-2,3-dihydro-1H-benzo[e][1,4]diazepin-3-yl)-nicotinamide

A mixture of racemic 3-amino-5-phenyl-1,3-dihydro-benzo[e][1,4]diazepin-2-one (1 g), O-benzotriazol-1-yl-N,N,N′,N′-tetramethyluronium hexafluorophosphate (1.51 g), triethylamine (0.83 ml) and 6-chloro-nicotinic acid (0.63 g) in dry DMF (20 ml) was stirred at room temperature for 1.5 h. Water (200 ml) was then added and the mixture stirred vigorously for 10 mins. The colourless precipitate was collected by filtration and dried (1.1 g)

¹H NMR (DMSO, δ) 5.50 (d, 1H) 7.28-7.71 (m, 10H) 8.42 (dd, 1H) 9.01 (d, 1H) 9.99 (d, 1H) 10.95 (s, 1H)

LC/MS RT=4.96 mins, ES+ 391,393

### Intermediate 16

### Thiomorpholine-1,1-dioxide

9.98 g of thiomorpholine and 14.8 g of triflic anhydride were stirred together in DCM at room temperature for 2 hours. The reaction was then partitioned between 1 M K₂CO_{3(aq)} and DCM. The organic layer was separated and dried by passing through a hydrophobic frit, then concentrated in vacuo. 13.82 g of the resultant oil was stirred with 85.2 g of oxone in 50 mL of methanol and 50 mL of water for 18 h at room temperature. The reaction was then filtered and washed with methanol and the filtrate concentrated. This was then partitioned between water and EtOAc and the aqueous layer washed 3 times with EtOAc. The combined organic extracts were then dried (MgSO₄) and concentrated to produce a white solid. This was then stirred at room temperature with 40 g of K₂CO₃ in 80 mL of methanol for 18 h. The methanol was then removed in vacuo and the remains partitioned between DCM and sat. K₂CO_{3(aq)}. The combined organic extracts were passed through a hydrophobic frit and concentrated in vacuo to produce the title compound, 3.51 g.

¹H NMR (CDCl₃, δ) 1.54 (s, 1H), 2.93-2.97 (m, 4H), 3.24-3.28 (m, 4H).

### Intermediate 17

### 5-{[(2-Methanesulfonyl-ethyl)-methyl-amino]-methyl}-furan-2-carboxylic acid ethyl ester

0.5 g of 5-chloromethyl-furan-2-carboxylic acid ethyl ester and 20 ml of 2 M methylamine solution in THF were stirred at room temperature for 5 days under nitrogen. The solution was then concentrated and purified by SPE. The resultant oil was heated at 200° C. in a microwave with 0.2 mL of methanesulfonyl-ethene in 3 mL of acetonitrile for 1 h. The solution was concentrated and purified by chromatography to produce the title compound as a colourless oil.

LC/MS RT=3.55 min, Found ES⁺=290

¹H NMR (CDCl₃, δ) 1.29 (t, 3H), 2.25 (s, 3H), 2.92-2.88 (m, 2H), 2.99 (s, 3H), 3.06-2.99 (t, 2H), 3.6 (s, 2H), 4.26 (q, 2H), 6.28 (d, 1H), 7.04 (d, 1H).

### Intermediate 18

### 5-Dimethylaminomethyl-furan-2-carboxylic acid

0.16 ml of a 2 M solution of dimethylamine was added to a stirred suspension of 19.2 mg of sodium hydride in 2 mL of DMF under a nitrogen atmosphere at room temperature for 30 min. Then a solution of 5-chloromethyl-furan-2-carboxylic acid ethyl ester in 2 mL of DMF was added dropwise over a period of 30 min. The reaction was then allowed to stir for 2 days. The solvent was then removed in vacuo and 5 mL of EtOH and 0.35 ml of 2 M NaOH added and stirred at 80° C. for 40 min. Upon return the reaction was acidified below pH 5.0 and the solvent removed in vacuo to produce the title compound to be hydrolysed and then used crude in the next stage

Intermediates 19-23 were prepared in an analogous manner and were used without characterisation in the next synthetic step

### Intermediate 19

### 5-Morpholin-4-ylmethyl-furan-2-carboxylic acid

### Intermediate 20

### 5-(1,1-Dioxo-1λ6-thiomorpholin-4-ylmethyl)-furan-2-carboxylic acid

### Intermediate 21

### 5-(4-Methyl-piperazin-1-ylmethyl)-furan-2-carboxylic acid

### Intermediate 22

### 5-(Piperidin-1-ylmethyl)-furan-2-carboxylic acid

### Intermediate 23

### 5-(Pyrrolidin-1-ylmethyl)-furan-2-carboxylic acid

### Intermediate 24

### 3-Cyclopropyl-1,3-dihydro[4,5-b]pyridin-2-one

A mixture of 2-chloro-3-nitro-pyridine (2 g), cyclopropylamine (1.13 ml) and potassium carbonate (3.48 g) in acetonitrile (30 ml) was stirred at room temperature for 18 h. The mixture was then partitioned between water and ethyl acetate. The dried extracts were evaporated giving a bright yellow solid (2.1 g)

This material was then hydrogenated at atmospheric pressure in ethanol (150 ml) over palladium on carbon catalyst (10%, 100 mg). When hydrogen uptake had ceased the mixture was filtered through celite and evaporated giving a dark gum (1.7 g)

This material was then dissolved in dry THF (40 ml) and was treated with carbonyl di-imidazole (2.2 g) at reflux for 2.5 h. The mixture was then partitioned between water and ethyl acetate. The dried organic extract was evaporated leaving a dark gum, which was crystallised from ethyl acetate/petrol giving a colourless solid (1.2 g)

¹H NMR (DMSO, δ) 0.97-1.04 (m, 4H) 2.92 (m, 1H) 6.97 (dd, 1H) 7.22 (dd, 1H) 7.92 (dd, 1H) 10.95 (brs, 1H)

### Intermediate 25

### 2-Morpholin-4-ylmethyl-furan-3-carboxylic acid methyl ester

A mixture of 2-chloromethyl-furan-3-carboxylic acid methyl ester (100 mg) and morpholine (0.08 ml) in acetonitrile (4 ml) was stirred at room temperature for 2 h. The mixture was then partitioned between dichloromethane and aqueous sodium bicarbonate solution. The dried organic layer was evaporated giving a yellow oil (75 mg)

¹H NMR (CDCl₃, δ) 2.57 (m, 4H) 3.74 (m, 4H) 3.86 (s, 3H) 3.97 (s, 2H) 6.70 (d, 1H) 7.38 (d, 1H)

### Intermediate 26

### 3-Morpholin-4-ylmethyl-benzoic acid methyl ester

This material was prepared as for Intermediate 25. The product was a colourless oil (210 mg)

¹H NMR (CDCl₃, δ) 2.43 (m, 4H) 3.53 (s, 2H) 3.70 (m, 4H) 3.91 (s, 3H) 7.39 (t, 1H) 7.42 (dd, 1H) 7.93 (dt, 1H) 7.99 (brs, 1H)

### Intermediate 27

### 5-Morpholin-4-ylmethyl-isoxazole-3-carboxylic acid methyl ester

5-Methyl-isoxazole-3-carboxylic acid methyl ester (200 mg), N-bromosuccinimide (252 mg) and benzoyl peroxide (30 mg) in dry chloroform (4 ml) was stirred and heated to 85 C for 5 h. The solution was cooled to room temperature and was treated with morpholine (0.27 ml). Stirring was continued for 20 h and the mixture was then partitioned between water and dichloromethane. The dried organic extract was evaporated and the residue purified on a silica gel SPE cartridge. Elution with dichloromethane followed by dichloromethane:ethanol:0.880 ammonia; 200:8:1 gave a colourless oil (50 mg)

¹H NMR (CDCl₃, δ) 2.46 (m, 4H) 3.64 (m, 4H) 3.67 (s, 2H) 3.90 (s, 3H) 6.55 (s, 1H)

Intermediates 28-30 were prepared in an analogous method to Intermediate 25

### Intermediate 28

### 3-Morpholin-4-ylmethyl-furan-2-carboxylic acid methyl ester

This compound was isolated as a yellow oil (189 mg)

¹H NMR (CDCl₃, δ) 2.45 (m, 4H) 3.65 (m, 4H) 3.71 (s, 2H) 3.85 (s, 3H) 6.56 (d, 1H) 7.45 (d, 1H)

### Intermediate 29

### 3-Morpholin-4-ylmethyl-thiophene-2-carboxylic acid methyl ester

This compound was isolated as yellow oil (197 mg).

¹H NMR (CDCl₃, δ) 2.50 (m, 4H) 3.69 (s, 2H) 3.72 (m, 4H) 3.86 (s, 3H) 6.90 (d, 1H) 7.64 (d, 1H)

### Intermediate 30

### 5-Morpholin-4-ylmethyl-thiophene-2-carboxylic acid methyl ester

This compound was isolated as a yellow oil (214 mg).

¹H NMR (CDCl₃, δ) 2.44 (m, 4H) 3.64 (m, 4H) 3.79 (s, 3H) 3.84 (s, 2H) 7.15 (d, 1H) 7.36 (d, 1H)

Intermediates 25-30 were hydrolysed to the corresponding carboxylic acids before use in the final coupling step of the synthetic sequence

### Intermediate 31

### 4-Fluoro-2-morpholin-4-yl-benzoic acid

2,4-Difluoro-benzoic acid (50 mg) and morpholine (0.03 ml) in acetonitrile (0.5 ml) were heated in a microwave at 200 C for 15 mins. The solvent was evaporated leaving a dark gum which was used without purification in the next synthetic step.

### Intermediate 32

### 4-Fluoro-2-piperidin-1-yl-benzoic acid

This was prepared in an analogous procedure to Intermediate 31.

Intermediates 33-5 were prepared in an analogous procedure to Intermediate 31 except that 2-fluoro-4-trifluoromethyl-benzoic acid was used.

### Intermediate 33

### 2-Pyrrolidin-1-yl-4-trifluoromethyl-benzoic acid

### Intermediate 34

### 2-Piperidin-1-yl-4-trifluoromethyl-benzoic acid

### Intermediate 35

### 2-Morpholin-4-yl-4-trifluoromethyl-benzoic acid

Intermediates 36 and 37 were prepared in an analogous procedure to Intermediate 31 except that 2-fluoro-5-trifluoromethyl-benzoic acid was used.

### Intermediate 36

### 2-Pyrrolidin-1-yl-5-trifluoromethyl-benzoic acid

### Intermediate 37

### 2-Morpholin-4-yl-5-trifluoromethyl-benzoic acid

Intermediates 38 and 39 were prepared in an analogous procedure to Intermediate 31 except that 4-cyano-2-fluoro-benzoic acid was used.

### Intermediate 38

### 4-Cyano-2-pyrrolidin-1-yl-benzoic acid

### Intermediate 39

### 4-Cyano-2-piperidin-1-yl-benzoic acid

### Example 1

### 6-(4-Methyl-piperazin-1-yl)-N-(2-oxo-5-phenyl-2,3-dihydro-1H-benzo[e][1,4]diazepin-3-yl)-nicotinamide

Intermediate 15 (50 mg) and N-methylpiperazine (0.022 ml) in acetonitrile (1 ml) containing triethylamine (0.027 ml) was heated in a microwave at 200° C. for 10 mins. The mixture was then partitioned between water and dichloromethane. The dried organic layer was evaporated and the residue purified on a silica gel SPE cartridge. Gradient elution with 5-10% methanol in dichloromethane gave a colourless solid (10 mg)

1H NMR (DMSO, d) 2.28 (s, 3H) 2.45 (m, 4H) 3.68 (m, 4H) 5.56 (d, 1H) 6.93 (d, 1H) 7.32-7.72 (m, 10H) 8.20 (dd, 1H) 8.82 (d, 1H) 9.42 (d, 1H) 10.94 (s, 1H)

RT=3.94 mins, ES+ 455

### Example 2

### 3,4,5,6-Tetrahydro-2H-[1,2′]bipyridinyl-5′-carboxylic acid (2-oxo-5-phenyl-2,3-dihydro-1H-benzo[e][1,4]diazepin-3-yl)-amide

This material was prepared as for Example 1 except that piperidine was used as the nucleophile. The product was a colourless solid (15 mg)

1H NMR (DMSO, d) 1.54-1.63 (brm, 6H) 3.65 (m, 4H) 5.48 (d, 1H) 6.86 (d, 1H) 7.25-7.65 (m, 10H) 8.11 (dd, 1H) 8.75 (d, 1H) 9.32 (d, 1H)

RT=4.54 mins, ES+ 440

### Example 3

### (S)-2-(1,1-Dioxo-1λ6-thiomorpholin-4-yl)-N-(2-oxo-5-phenyl-2,3-dihydro-1H-benzo[e][1,4]diazepin-3-yl-benzamide

(S)-3-Amino-5-phenyl-1,3-dihydro-benzo[e][1,4]diazepin-2-one (100 mg), O-benzotriazol-1-yl-N,N,N′,N′-tetramethyluronium hexafluorophosphate (150 mg), 2-(1,1-dioxo-1λ6-thiomorpholin-4-yl)-benzoic acid (102 mg) and triethylamine (0.083 ml) in dry DMF (1 ml) was stirred at room temperature for 1 h. Water (10 ml) was then added and stirring continued for 10 mins. The colourless precipitate was collected by filtration and then partitioned between dichloromethane and water. The dried organic phase was evaporated and the residue purified on a silica gel SPE cartridge. Elution with ethyl acetate:petrol 1:1 gave the title compound as a colourless solid (140 mg)

¹H NMR (DMSO, δ) 3.49 (brs, 8H) 5.48 (d, 1H) 7.31-7.95 (m, 13H) 10.86 (d, 1H) 11.18 (s, 1H)

### Example 4

### (S)-2-Chloro-4-morpholin-4-yl-N-(2-oxo-5-phenyl-2,3-dihydro-1H-benzo[e][1,4]diazepin-3-yl)-benzamide

This material was prepared as for Example 3 except that 2-chloro-4-morpholin-4-yl-benzoic acid (86 mg) was used. The title compound was a colourless solid (112 mg).

¹H NMR (DMSO, δ) 3.21 (m, 4H) 3.70 (t, 4H) 5.36 (d, 1H) 6.90-6.97 (m, 2H) 7.21-7.66 (m, 10H) 9.21 (d, 1H) 10.86 (s, 1H)

### Example 5

### (S)-2-(1,1-Dioxo-4-oxy-1λ6-thiomorpholin-4-yl)-4-fluoro-(2-oxo-5-phenyl-2,3-dihydro-1H-benzo[e][1,4]diazepin-3-yl-benzamide

This material was prepared as for Example 3 except that 2-(1,1-dioxo-4-oxy-1λ6-thiomorpholin-4-yl)-benzoic acid (Intermediate 14, 30 mg) was used. The title compound was a colourless solid (29 mg).

1H NMR (DMSO, d) 3.32-3.98 (m, 8H) 5.34 (d, 1H) 6.99 (dt, 1H) 7.16-7.65 (m, 11H) 9.51 (d, 1H) 10.98 (s, 1H)

RT=5.09 mins, ES+ 523

### Example 6

### (S)-5-Chloro-2-(1,1-dioxo-1λ6-thiomorpholin-4-yl)-N-(2-oxo-5-phenyl-2,3-dihydro-1H-benzo[e][1,4]diazepin-3-yl)-benzamide

This material was prepared as for Example 3 except that 5-Chloro-2-(1,1-dioxo-1λ6-thiomorpholin-4-yl)-benzoic acid (Intermediate 11, 58 mg) was used. The title compound was a colourless solid (70 mg).

1H NMR (DMSO, d) 3.54 (s, 8H) 5.53 (d, 1H) 7.37-7.75 (m, 1H) 7.90 (d, 1H) 10.84 (d, 1H) 11.24 (s, 1H)

RT=5.38 mins, ES+ 523,525

### Example 7

### (S)-2-(1,1-Dioxo-1λ6-thiomorpholin-4-yl)-5-fluoro-N-(2-oxo-5-phenyl-2,3-dihydro-1H-benzo[e][1,4]diazepin-3-yl)-benzamide

This material was prepared as for Example 3 except that 5-Fluoro-2-(1,1-dioxo-1λ6-thiomorpholin-4-yl)-benzoic acid (Intermediate 12, 54 mg) was used. The title compound was a colourless solid (70 mg).

1H NMR (DMSO, d) 3.49 (m, 8H) 5.47 (d, 1H) 7.34-7.69 (m, 12H) 11.12 (d, 1H) 11.20 (s, 1H)

RT=5.19 mins, ES+ 507

### Example 8

### (S)-5-(4-Methyl-piperazin-1-ylmethyl)-furan-2-carboxylic acid (2-oxo-5-phenyl-2,3-dihydro-1H-benzo[e][1,4]diazepin-3-yl)-amide

This material was prepared as for Example 3 except that 5-(4-Methyl-piperazin-1-ylmethyl)-furan-2-carboxylic acid (Intermediate 21) was used. The title compound was a colourless solid (15 mg).

1H NMR (CDCl₃, d) 2.23 (s, 3H), 2.43-2.51 (m, 8H), 3.56 (s, 2H), 5.65 (d, 1H), 6.29 (d, 1H), 7.05-7.51 (m, 11H), 7.92 (d, 1H).

RT=4.10 mins, ES+ 458

### Example 9

### (S)-5-Pyrrolidin-1-ylmethyl-furan-2-carboxylic acid (2-oxo-5-phenyl-2,3-dihydro-1H-benzo[e][1,4]diazepin-3-yl)-amide

This material was prepared as for Example 3 except that 5-(pyrrolidin-1-ylmethyl)-furan-2-carboxylic acid (Intermediate 23) was used. The title compound was a colourless solid (52 mg).

1H NMR (CDCl₃, d) 1.76-1.77 (m, 4H), 2.60-2.62 (m, 4H), 3.71 (s, 2H), 5.64 (d, 1H), 6.31 (d, 1H), 7.05-7.50 (m, 10H), 7.98 (d, 1H), 8.04 (s, 1H).

RT=4.09 mins, ES+ 403

### Example 10

### (S)-5-Piperidin-1-ylmethyl-furan-2-carboxylic acid (2-oxo-5-phenyl-2,3-dihydro-1H-benzo[e][1,4]diazepin-3-yl)-amide

This material was prepared as for Example 3 except that 5-(piperidin-1-ylmethyl)-furan-2-carboxylic acid (Intermediate 22) was used. The title compound was a colourless solid (21 mg).

1H NMR (CDCl₃, d) 1.36-1.45 (m, 2H), 1.53-1.60 (m, 4H), 2.45-2.55 (m, 4H), 3.62 (s, 2H), 5.65 (d, 1H), 6.34 (d, 1H), 7.06-5.52 (m, 10H), 7.81-7.89 (m, 1H), 7.96 (d, 1H).

RT=4.16 mins, ES+ 443

### Example 11

### (S)-5-Dimethylaminomethyl-furan-2-carboxylic acid (2-oxo-5-phenyl-2,3-dihydro-1H-benzo[e][1,4]diazepin-3-yl)-amide

This material was prepared as for Example 3 except that 5-dimethylaminomethyl-furan-2-carboxylic acid (Intermediate 18) was used. The title compound was a colourless solid (5 mg).

1H NMR (DMSO, d) 2.35 (s, 6H), 3.69 (s, 2H), 5.56 (d, 1H), 6.65 (d, 1H), 7.48-7.85 (m, 10H), 9.1 (d, 1H), 11.13 (s, 1H).

RT=4.09 mins, ES+ 403

### Example 12

### (S)-4-Fluoro-N-(2-oxo-5-phenyl-2,3-dihydro-1H-benzo[e][1,4]diazepin-3-yl)-2-piperidin-1-yl-benzamide

This material was prepared as for Example 3 except that 4-fluoro-2-piperidin-1-yl-benzoic acid (Intermediate 32) was used. The title compound was a colourless solid (58 mg).

1H NMR (DMSO, d) 1.62-1.67 (m, 2H) 1.91-1.99 (m, 4H) 3.08-3.16 (m, 4H) 5.56 (d, 1H) 7.15-7.79 (m, 11H) 8.10-8.13 (m, 1H) 11.08 (s and d, 2H)

RT=6.02 mins, ES+ 457

### Example 13

### (S)-4-Fluoro-2-morpholino-4-yl-N-(2-oxo-5-phenyl-2,3-dihydro-1H-benzo[e][1,4]diazepin-3-yl)-benzamide

This material was prepared as for Example 3 except that 4-fluoro-2-morpholin-4-yl-benzoic acid (Intermediate 31) was used. The title compound was a colourless solid (19 mg).

1H NMR (DMSO, d) 2.94-3.00 (m, 4H) 3.71-3.82 (m, 4H) 5.35 (d, 1H) 6.98-7.85 (m, 12H) 10.52 (d, 1H) 10.90 (s, 1H)

RT=5.34 mins, ES+ 459

### Example 14

### (S)-4-Cyano-N-(2-oxo-5-phenyl-2,3-dihydro-1H-benzo[e][1,4]diazepin-3-yl)-2-pyrrolidin-1-yl-benzamide

This material was prepared as for Example 3 except that 4-cyano-2-pyrrolidin-1-yl-benzoic acid (Intermediate 38) was used. The title compound was a colourless solid (13 mg).

1H NMR (DMSO, d) 1.87 (brs, 4H) 3.29 (brs, 4H) 5.37 (d, 1H) 7.01-7.65 (m, 12H) 9.60 (d, 1H) 10.88 (s, 1H)

RT=5.45 mins, ES+ 450

### Example 15

### (S)-4-Cyano-N-(2-oxo-5-phenyl-2,3-dihydro-1H-benzo[e][1,4]diazepin-3-yl)-piperidine-1-yl-benzamide

This material was prepared as for Example 3 except that 4-cyano-2-piperidin-1-yl-benzoic acid (Intermediate 39) was used. The title compound was a colourless solid (27 mg).

1H NMR (DMSO, d) 1.32-1.36 (m, 2H) 1.58-1.67 (m, 4H) 2.81-2.89 (m, 4H) 5.25 (d, 1H) 7.10-7.83 (m, 12H) 10.70 (d, 1H) 10.81 (s, 1H)

RT=5.88 mins, ES+ 464

### Example 16

### (S)—N-(2-Oxo-5-phenyl-2,3-dihydro-1H-benzo[e][1,4]diazepin-3-yl)-2-pyrrolidin-1-yl-4-trifluoromethyl-benzamide

This material was prepared as for Example 3 except that 2-pyrrolidin-1-yl-4-trifluoromethyl-benzoic acid (Intermediate 33) was used. The title compound was a colourless solid (5 mg).

1H NMR (DMSO, d) 1.89-1.92 (brs, 4H) 3.29-3.32 (brs, 4H) 5.40 (d, 1H) 6.88 (s, 1H) 6.94 (d, 1H) 7.24-7.67 (m, 10H) 9.56 (d, 1H) 10.89 (s, 1H)

RT=5.91 mins, ES+ 493

### Example 17

### (S)—N-(2-Oxo-5-phenyl-2,3-dihydro-1H-benzo[e][1,4]diazepin-3-yl)-2-piperidin-1-yl-4-trifluoromethyl-benzamide

This material was prepared as for Example 3 except that 2-piperidin-1-yl-4-trifluoromethyl-benzoic acid (Intermediate 34) was used. The title compound was a colourless solid (14 mg).

1H NMR (DMSO, d) 1.53-1.57 (m, 2H) 1.80-1.91 (m, 4H) 3.00-3.14 (m, 4H) 5.46 (d, 1H) 7.30-7.72 (m, 11H) 8.09 (d, 1H) 10.98 (d, 1H) 10.99 (s, 1H)

RT=6.39 mins, ES+ 507

### Example 18

### (S)-2-Morpholin-4-yl-N-(2-oxo-5-phenyl-2,3-dihydro-1H-benzo[e][1,4]diazepin-3-yl)-4-trifluoromethyl-benzamide

This material was prepared as for Example 3 except that 2-morpholin-4-yl-4-trifluoromethyl-benzoic acid (Intermediate 35) was used. The title compound was a colourless solid (14 mg).

1H NMR (DMSO, d) 3.18-3.24 (m, 4H) 3.90-3.96 (m, 4H) 5.52 (d, 1H) 7.36-8.10 (m, 12H) 10.59 (d, 1H) 11.10 (s, 1H)

RT=5.72 mins, ES+ 509

### Example 19

### (S)—N-(2-Oxo-5-phenyl-2,3-dihydro-1H-benzo[e][1,4]diazepin-3-yl)-2-pyrrolidin-1-yl-5-trifluoromethyl-benzamide

This material was prepared as for Example 3 except that 2-pyrrolidin-1-yl-5-trifluoromethyl-benzoic acid (Intermediate 36) was used. The title compound was a colourless solid (8 mg).

1H NMR (DMSO, d) 2.00-2.02 (brs, 4H) 3.40-3.43 (brs, 4H) 5.48 (d, 1H) 6.90 (d, 1H) 7.34-7.74 (m, 11H) 9.71 (d, 1H) 10.98 (s, 1H)

RT=5.84 mins, ES+ 493

### Example 20

### (S)-2-Morpholin-4-yl-N-(2-oxo-5-phenyl-2,3-dihydro-1H-benzo[e][1,4]diazepin-3-yl)-5-trifluoromethyl-benzamide

This material was prepared as for Example 3 except that 2-morpholin-4-yl-5-trifluoromethyl-benzoic acid (Intermediate 37) was used. The title compound was a colourless solid (19 mg).

1H NMR (DMSO, d) 3.13-3.18 (m, 4H) 3.85-3.90 (m, 4H) 5.46 (d, 1H) 7.30-7.69 (m, 10H) 7.88 (dd, 1H) 8.04 (d, 1H) 10.37 (d, 1H) 11.04 (s, 1H)

RT=5.72 mins, ES+ 509

### Example 21

### (S)-2-Morpholin-4-yl-N-(2-oxo-5-phenyl-2,3-dihydro-1H-benzo[e][1,4]diazepin-3-yl)-nicotinamide

This material was prepared as for Example 3 except that 2-morpholin-4-yl-nicotinic acid was used. The title compound was a colourless solid (45 mg).

1H NMR (DMSO, d) 3.30-3.36 (m, 4H) 3.82-3.85 (m, 4H) 5.45 (d, 1H) 7.14-7.17 (m, 1H) 7.19-7.71 (m, 9H) 8.07 (dd, 1H) 8.44 (dd, 1H) 10.00 (d, 1H) 11.05 (s, 1H)

RT=4.86 mins, ES+ 442

### Example 22

### (S)-2-(1,1-Dioxo-1λ6-thiomorpholin-4-yl)-N-(2-oxo-5-phenyl-2,3-dihydro-1H-benzo[e][1,4]diazepin-3-yl)-nicotinamide

This material was prepared as for Example 3 except that 2-(1,1-dioxo-1λ6-thiomorpholin-4-yl)-nicotinic acid (Intermediate 3) was used. The title compound was a colourless solid (10 mg).

1H NMR (DMSO, d) 3.25 (t, 2H) 3.40 (t, 2H) 3.75-3.88 (m, 4H) 5.47 (d, 1H) 6.67-6.72 (m, 1H) 7.28-7.67 (m, 8H) 8.24-8.38 (m, 3H) 9.56 (d, 1H) 10.92 (s, 1H)

RT=4.43 mins, ES+ 508

### Example 23

### (S)-2-(1,1-Dioxo-1λ6-thiomorpholin-4-yl)-3-methyl-N-(2-oxo-5-phenyl-2,3-dihydro-1H-benzo[e][1,4]diazepin-3-yl)-benzamide

This material was prepared as for Example 3 except that 2-(1,1-dioxo-1λ6-thiomorpholin-4-yl)-3-methyl-benzoic acid (Intermediate 4) was used. The title compound was a colourless solid (65 mg).

1H NMR (DMSO, d) 2.36 (s, 3H) 3.24 (brs, 4H) 3.49 (brs, 4H) 5.43 (d, 1H) 7.11-7.68 (m, 12H) 9.61 (d, 1H) 10.99 (s, 1H)

RT=5.04 mins, ES+ 503

### Example 24

### (S)-2-(1,1-Dioxo-1λ6-thiomorpholin-4-yl)-4-methyl-N-(2-oxo-5-phenyl-2,3-dihydro-1H-benzo[e][1,4]diazepin-3-yl)-benzamide

This material was prepared as for Example 3 except that 2-(1,1-dioxo-1λ6-thiomorpholin-4-yl)-4-methyl-benzoic acid (Intermediate 5) was used. The title compound was a colourless solid (72 mg).

1H NMR (DMSO, d) 2.39 (s, 3H) 3.44-3.54 (brm, 8H) 5.46 (d, 1H) 7.14 (d, 1H) 7.31-7.69 (m, 10H) 7.86 (d, 1H) 10.94 (d, 1H) 11.17 (s, 1H)

RT=5.20 mins, ES+ 503

### Example 25

### (S)-2-(1,1-Dioxo-1λ6-thiomorpholin-4-yl)-6-methyl-N-(2-oxo-5-phenyl-2,3-dihydro-1H-benzo[e][1,4]diazepin-3-yl)-benzamide

This material was prepared as for Example 3 except that 2-(1,1-dioxo-1λ6-thiomorpholin-4-yl)-6-methyl-benzoic acid (Intermediate 6) was used. The title compound was a colourless solid (32 mg).

1H NMR (DMSO, d) 2.27 (s, 3H) 3.24-3.27 (m, 4H) 3.41-3.43 (m, 4H) 5.56 (d, 1H) 7.03 (d, 1H) 7.11 (d, 1H) 7.25-7.68 (m, 10H) 9.44 (d, 1H) 10.96 (s, 1H)

RT=5.03 mins, ES+ 503

### Example 26

### (S)-2-Chloro-6-(1,1-dioxo-1,6-thiomorpholin-4-yl)-N-(2-oxo-5-phenyl-2,3-dihydro-1H-benzo[e][1,4]diazepin-3-yl)-benzamide

This material was prepared as for Example 3 except that 2-chloro-6-(1,1-dioxo-1λ6-thiomorpholin-4-yl)-benzoic acid (Intermediate 10) was used. The title compound was a colourless solid (51 mg).

1H NMR (DMSO, d) 3.43-3.47 (m, 4H) 3.59-3.61 (m, 4H) 5.63 (d, 1H) 7.39-7.83 (m, 12H) 9.86 (d, 1H) 11.14 (s, 1H)

RT=5.07 mins, ES+ 523, 525

### Example 27

### (S)-3-Cyclopropyl-2-oxo-2,3-dihydro-imidazo[4,5-b]pyridine-1-carboxylic acid (2-oxo-5-phenyl-2,3-dihydro-1H-benzo[e][1,4]diazepin-3-yl)-amide

3-Cyclopropyl-1,3-dihydro[4,5-b]pyridin-2-one (Intermediate 24, 35 mg), triethylamine (0.028 ml) and triphosgene (20 mg) were stirred at room temperature in dichloromethane (3 ml) for 1 h. (S)-3-Amino-5-phenyl-1,3-dihydro-benzo[e][1,4]diazepin-2-one (50 mg) was then added, and stirring continued for 18 h. The solvent was evaporated and the residue purified on a silica gel SPE cartridge. Elution with dichloromethane:ethanol:0.880 ammonia; 200:8:1 gave a colourless solid (3 mg)

1H NMR (DMSO, d) 0.88-1.09 (m, 4H) 2.92 (m, 1H) 5.25 (d, 1H) 7.06-7.71 (m, 10H) 8.08 (m, 2H) 9.94 (d, 1H) 11.08 (s, 1H)

RT=4.90 mins, ES+ 453

### Example 28

### (S)-3-(4-Methyl-piperazine-1-sulfonyl)-N-(2-oxo-5-phenyl-2,3-dihydro-1H-benzo[e][1,4]diazepin-3-yl)-benzamide

This material was prepared as for Example 3 except that 3-(4-methyl-piperazine-1-sulfonyl)-benzoic acid (Intermediate 7) was used. The title compound was a pale yellow solid (23 mg).

1H NMR (CDCl3, d) 2.19 (s, 3H), 2.39-2.43 (m, 4H), 2.95-3.05 (m, 4H), 5.68 (d, 1H), 6.5 (s, 1H), 7.13 (t, 2H), 7.19 (s, 1H), 7.32-7.83 (m, 8H), 8.08-8.11 (m, 2H), 8.28-8.29 (m, 1H).

RT=4.25 mins, ES+ 518

### Example 29

### (S)-4-(4-Methyl-piperazin-1-yl)-N-(2-oxo-5-phenyl-2,3-dihydro-1H-benzo[e][1,4]diazepin-3-yl)-benzamide

This material was prepared as for Example 3 except that 4-(4-methyl-piperazine-1-yl)-benzoic acid was used. The title compound was a colourless solid (46 mg).

1H NMR (CDCl3, d) 2.30 (s, 3H), 2.50-2.54 (m, 4H), 3.26-3.30 (m, 4H), 5.70 (d, 1H), 6.86 (d, 2H), 7.14 (t, 1H), 7.17-7.50 (m, 8H), 7.74 (d, 1H), 7.80 (d, 2H), 8.25-8.40 (m, 1H).

RT=4.16 mins, ES+ 454

### Example 30

### (S)—N-(2-Oxo-5-phenyl-2,3-dihydro-1H-benzo[e][1,4]diazepin-3-yl)-3-(piperidine-1-sulfonyl)-benzamide

This material was prepared as for Example 3 except that 3-piperidine-1-sulfonyl-benzoic acid (Intermediate 8) was used. The title compound was a colourless solid (35 mg).

1H NMR (CDCl3, d) 1.35-1.38 (m, 2H), 1.57-1.65 (m, 4H), 2.91-2.99 (m, 4H), 5.70 (d, 1H), 7.14 (t, 2H), 7.19 (s, 2H), 7.31-7.84 (m, 7H), 8.04-8.12 (m, 2H), 8.28-8.29 (m, 1H), 8.41 (s, 1H).

RT=5.47 mins, ES+ 503

### Example 31

### (S)-3-(Morpholine-4-sulfonyl)-N-(2-oxo-5-phenyl-2,3-dihydro-1H-benzo[e][1,4]diazepin-3-yl)-benzamide

This material was prepared as for Example 3 except that 3-(morpholine-4-sulfonyl)-benzoic acid (Intermediate 9) was used. The title compound was a colourless solid (29 mg).

1H NMR (CDCl3, d) 2.97-3.00 (m, 4H), 3.66-3.70 (m, 4H), 5.68 (d, 1H), 7.10-8.18 (m, 13H), 8.29-8.31 (m, 2H).

RT=5.06 mins, ES+ 505

### Example 32

### (S)-5-Morpholin-4-ylmethyl-furan-2-carboxylic acid (2-oxo-5-phenyl-2,3-dihydro-1H-benzo[e][1,4]diazepin-3-yl)-amide

This material was prepared as for Example 3 except that 5-morpholin-4-ylmethyl-furan-2-carboxylic acid (Intermediate 19) was used. The title compound was a colourless solid (35 mg).

1H NMR (CDCl3, d) 2.46-2.49 (m, 4H), 3.55 (s, 2H), 3.66-3.70 (m, 4H), 5.65 (d, 1H), 6.30 (d, 1H), 7.06-7.51 (m, 10H), 7.95 (d, 1H), 8.38 (s, 1H).

RT=4.28 mins, ES+ 445

### Example 33

### (S)-5-Hydroxymethyl-furan-2-carboxylic acid (2-oxo-5-phenyl-2,3-dihydro-1H-benzo[e][1,4]diazepin-3-yl)-amide

This material was prepared as for Example 3 except that the hydrolysis product of 5-chloromethyl-furan-2-carboxylic acid ethyl ester was used. The title compound was a colourless solid (48 mg).

1H NMR (CDCl3, d) 2.78 (s, 1H), 4.55-4.56 (m, 2H), 5.63 (d, 1H), 6.25 (d, 1H), 7.00 (d, 1H), 7.09 (t, 2H), 7.15-7.49 (m, 7H), 8.10 (d, 1H), 8.46 (s, 1H).

RT=4.54 mins, ES+ 376

### Example 34

### (S)-5-(1,1-Dioxo-1λ6-thiomorpholin-4-ylmethyl)-furan-2-carboxylic acid (2-oxo-5-phenyl-2,3-dihydro-1H-benzo[e][1,4]diazepin-3-yl)-amide

This material was prepared as for Example 3 except that 5-(1,1-Dioxo-1□6-thiomorpholin-4-ylmethyl)-furan-2-carboxylic acid (Intermediate 20) was used. The title compound was a colourless solid (192 mg).

1H NMR (CDCl3, d) 3.00-3.10 (m, 8H), 3.68 (s, 2H), 5.65 (d, 1H), 6.32 (d, 1H), 7.06-7.50 (m, 10H), 7.95 (d, 1H), 8.08-8.16 (s, 1H).

RT=4.65 mins, ES+ 493

### Example 35

### (S)-2-Chloro-4-(1,1-dioxo-1λ6-thiomorpholin-4-yl)-N-(2-oxo-5-phenyl-2,3-dihydro-1H-benzo[e][1,4]diazepin-3-yl)-benzamide

This material was prepared as for Example 3 except that 2-chloro-4-(1,1-dioxo-1λ6-thiomorpholin-4-yl)-benzoic acid (Intermediate 1) was used. The title compound was a colourless solid (41 mg).

1H NMR (DMSO, d) 3.15 (brs, 4H) 3.92 (brs, 4H) 5.41 (d, 1H) 7.10-7.68 (m, 12H) 9.26 (d, 1H) 10.92 (s, 1H)

RT=4.70 mins, ES+ 523, 525

### Example 36

### (S)-2-Chloro-5-(1,1-dioxo-1λ6-thiomorpholin-4-yl)-N-(2-oxo-5-phenyl-2,3-dihydro-1H-benzo[e][1,4]diazepin-3-yl)-benzamide

This material was prepared as for Example 3 except that 2-chloro-5-(1,1-dioxo-1λ6-thiomorpholin-4-yl)-benzoic acid (Intermediate 2) was used. The title compound was a colourless solid (69 mg).

1H NMR (DMSO, d) 3.14 (brs, 4H) 3.81 (brs, 4H) 5.37 (d, 1H) 7.08-7.63 (m, 12H) 9.56 (d, 1H) 10.84 (s, 1H)

RT=4.76 mins, ES+ 523,525

### Example 37

### (S)-5-{[(2-Methanesulfonyl-ethyl)-methyl-amino]-methyl}-furan-2-carboxylic acid (2-oxo-5-phenyl-2,3-dihydro-1H-benzo[e][1,4]diazepin-3-yl-amide

This material was prepared as for Example 3 except that 5-{[(2-methanesulfonyl-ethyl)-methyl-amino]-methyl}-furan-2-carboxylic acid ethyl ester (Intermediate 17) was used. The title compound was a colourless solid (87 mg).

1H NMR (DMSO, d) 2.05 (s, 3H), 2.61 (t, 2H), 2.84 (s, 3H), 3.12 (t, 2H), 3.48 (s, 2H), 5.21 (d, 1H), 6.34 (d, 1H), 7.05-7.39 (m, 9H), 7.50 (td, 1H), 8.77 (d, 1H), 10.78 (s, 1H).

RT=4.78 mins, ES+ 495

### Example 38

### (S)-2-Pyridin-3-yl-thiazole-4-carboxylic acid (2-oxo-5-phenyl-2,3-dihydro-1H-benzo[e][1,4]diazepin-3-yl)-amide

This material was prepared as for Example 3 except that 2-pyridin-3-yl-thiazole-4-carboxylic acid was used. The title compound was a colourless solid (55 mg).

1H NMR (DMSO, d) 5.64 (d, 1H) 7.48-7.86 (m, 10H) 8.66 (dt, 1H) 8.73 (s, 1H) 8.93 (dd, 1H) 9.31 (d, 1H) 9.47 (d, 1H) 11.28 (s, 1H)

RT=4.70 mins, ES+ 440

### Example 39

### (S)-2-Pyridin-4-yl-thiazole-4-carboxylic acid (2-oxo-5-phenyl-2,3-dihydro-1H-benzo[e][1,4]diazepin-3-yl)-amide

This material was prepared as for Example 3 except that 2-pyridin-4-yl-thiazole-4-carboxylic acid was used. The title compound was a colourless solid (54 mg).

1H NMR (DMSO, d) 5.36 (d, 1H) 7.19-7.58 (m, 9H) 7.96 (dd, 2H) 8.53 (s, 1H) 8.69 (dd, 2H) 9.02 (d, 1H) 11.01 (s, 1H)

RT=4.69 mins, ES+ 440

### Example 40

### (S)-4-Methyl-2-pyrazin-2-yl-thiazole-5-carboxylic acid (2-oxo-5-phenyl-2,3-dihydro-1H-benzo[e][1,4]diazepin-3-yl)-amide

This material was prepared as for Example 3 except that 4-methyl-2-pyrazin-2-yl-thiazole-5-carboxylic acid was used. The title compound was a colourless solid (67 mg).

1H NMR (DMSO, d) 2.56 (s, 3H) 5.25 (d, 1H) 7.10-7.49 (m, 9H) 8.58-8.63 (s+dd, 2H) 9.16 (d, 1H) 9.38 (d, 1H) 10.78 (s, 1H)

RT=4.82 mins, ES+ 455

### Example 41

### (S)-2-Morpholin-4-ylmethyl-furan-3-carboxylic acid (2-oxo-5-phenyl-2,3-dihydro-1H-benzo[e][1,4]diazepin-3-yl)-amide

This material was prepared as for Example 3 except that 2-morpholin-4-ylmethyl-furan-3-carboxylic acid (Intermediate 25) was used. The title compound was a colourless solid (24 mg).

1H NMR (DMSO, d) 2.58 (brm, 4H) 3.67 (brm, 4H) 3.91 (s, 2H) 5.45 (d, 1H) 6.88 (d, 1H) 7.33-7.75 (m, 10H) 10.95 (s, 1H) 11.01 (d, 1H)

RT=5.04 mins, ES+ 445

### Example 42

### (S)-3-Morpholin-4-ylmethyl-N-(2-oxo-5-phenyl-2,3-dihydro-1H-benzo[e][1,4]diazepin-3-yl)-benzamide

This material was prepared as for Example 3 except that 3-morpholin-4-ylmethyl-benzoic acid (Intermediate 26) was used. The title compound was a colourless solid (24 mg).

1H NMR (DMSO, d) 2.39 (brm, 4H) 3.55 (s, 2H) 3.60 (brm, 4H) 5.51 (d, 1H) 7.28-7.71 (m, 11H) 7.93 (s, 1H) 7.97 (s, 1H) 9.50 (d, 1H) 10.93 (s, 1H)

RT=4.86 mins, ES+ 455

### Example 43

### (S)-5-Morpholin-4-ylmethyl-isoxazole-3-carboxylic acid (2-oxo-5-phenyl-2,3-dihydro-1H-benzo[e][1,4]diazepin-3-yl)-amide

This material was prepared as for Example 3 except that 5-morpholin-4-ylmethyl-isoxazole-3-carboxylic acid (Intermediate 27) was used. The title compound was a colourless solid (11 mg).

1H NMR (DMSO, d) 2.93 (m, 4H) 3.46 (m, 4H) 3.66 (brs, 2H) 5.26 (d, 1H) 6.77 (s, 1H) 7.13-7.38 (m, 9H) 9.17 (d, 1H) 10.90 (s, 1H)

RT=4.75 mins, ES+ 446

### Example 44

### (S)-3-Morpholin-4-ylmethyl-furan-2-carboxylic acid (2-oxo-5-phenyl-2,3-dihydro-1H-benzo[e][1,4]diazepin-3-yl)-amide

This material was prepared as for Example 3 except that 3-morpholin-4-ylmethyl-furan-2-carboxylic acid (Intermediate 28) was used. The title compound was a colourless solid (20 mg).

1H NMR (DMSO, d) 2.52 (brm, 4H) 3.62 (brs, 4H) 3.67 (m, 2H) 5.39 (d, 1H) 6.67 (d, 1H) 7.25-7.71 (m, 9H) 7.84 (d, 1H) 10.93 (s, 1H) 11.34 (d, 1H)

RT=4.96 mins, ES+ 445

### Example 45

### (S)-5-Pyridin-2-yl-thiophene-2-carboxylic acid (2-oxo-5-phenyl-2,3-dihydro-1H-benzo[e][1,4]diazepin-3-yl)-amide

This material was prepared as for Example 3 except that 5-pyridin-2-yl-thiophene-2-carboxylic acid was used. The title compound was a colourless solid (32 mg).

1H NMR (DMSO, d) 5.58 (d, 1H) 7.37-7.77 (m, 10H) 7.96-7.99 (m, 2H) 8.10 (d, 1H) 8.32 (d, 1H) 8.67 (d, 1H) 9.81 (d, 1H) 11.03 (s, 1H)

RT=4.91 mins, ES+ 439

### Example 46

### (S)-2-Methyl-4-(morpholin-4-sulfonyl)-furan-3-carboxylic acid (2-oxo-5-phenyl-2,3-dihydro-1H-benzo[e][1,4]diazepin-3-yl)-amide

This material was prepared as for Example 3 except that 2-methyl-4-(morpholin-4-sulfonyl)-furan-3-carboxylic acid was used. The title compound was a colourless solid (75 mg).

1H NMR (DMSO, d) 2.77 (s, 3H) 3.26 (m, 4H) 3.85 (m, 4H) 5.60 (d, 1H) 7.43-7.83 (m, 9H) 8.23 (s, 1H) 9.68 (d, 1H) 11.07 (s, 1H)

RT=4.90 mins, ES+ 509

### Example 47

### (S)-6-Morpholin-4-yl-N-(2-oxo-5-phenyl-2,3-dihydro-1H-benzo[e][1,4]diazepin-3-yl)-nicotinamide

This material was prepared as for Example 3 except that 6-morpholin-4-nicotinic acid was used. The title compound was a colourless solid (28 mg).

1H NMR (DMSO, d) 3.58-3.61 (m, 4H) 3.70-3.73 (m, 4H) 5.51 (d, 1H) 6.89 (d, 1H) 7.24-7.71 (m, 9H) 8.19 (dd, 1H) 8.80 (d, 1H) 9.39 (d, 1H) 10.89 (s, 1H)

RT=4.59 mins, ES+ 442

### Example 48

### (S)-3-Morpholin-4-ylmethyl-thiophene-2-carboxylic acid (2-oxo-5-phenyl-2,3-dihydro-1H-benzo[e][1,4]diazepin-3-yl)-amide

This material was prepared as for Example 3 except that 3-morpholin-4-ylmethyl-thiophene-2-carboxylic acid (Intermediate 29) was used. The title compound was a colourless solid (34 mg).

1H NMR (DMSO, d) 2.43 (m, 4H) 3.59 (m, 4H) 3.70 (s, 2H) 5.45 (d, 1H) 7.05 (d, 1H) 7.24-7.70 (m, 9H) 8.05 (d, 1H) 9.54 (d, 1H) 10.92 (s, 1H)

RT=5.02 mins, ES+ 461

### Example 49

### (S)-5-Morpholin-4-ylmethyl-thiophene-2-carboxylic acid (2-oxo-5-phenyl-2,3-dihydro-1H-benzo[e][1,4]diazepin-3-yl)-amide

This material was prepared as for Example 3 except that 5-morpholin-4-ylmethyl-thiophene-2-carboxylic acid (Intermediate 30) was used. The title compound was a colourless solid (41 mg).

1H NMR (DMSO, d) 2.28 (brm, 4H) 3.38 (brm, 4H) 3.56 (s, 2H) 5.16 (d, 1H) 6.90 (d, 1H) 7.04-7.44 (m, 9H) 7.52 (d, 1H) 10.68 (s, 1H) 11.82 (d, 1H)

RT=5.33 mins, ES+ 461

### Example 50

### 2-Morpholin-4-yl-N-(2-oxo-5-phenyl-2,3-dihydro-1H-benzo[e][1,4]diazepin-3-yl)-benzamide

This material was prepared as for Intermediate 15 except that 2-morpholin-4-yl-benzoic acid (49 mg) was used. The product was a colourless solid (33 mg)

1H NMR (DMSO, d) 3.01-3.12 (m, 4H) 3.86-3.93 (m, 4H) 5.44 (d, 1H) 7.21-7.71 (m, 12H) 7.93 (dd, 1H) 10.99 (d, 1H) 11.02 (s, 1H)

RT=5.47, ES+ 441

### Example 51

### (S)-5-Phenyl-oxazole-4-carboxylic acid (2-oxo-5-phenyl-2,3-dihydro-1H-benzo[e][1,4]diazepin-3-yl)-amide

(S)-3-Amino-5-phenyl-1,3-dihydro-benzo[e][1,4]diazepin-2-one (60 mg), triethylamine (0.037 ml) and 5-phenyl-oxazole-4-carbonyl chloride (50 mg) in THF (3 ml) were stirred at room temperature for 2 h. The mixture was then partitioned between water and dichloromethane. The dried organic phase was evaporated and the residue purified on a silica gel SPE cartridge. Elution with dichloromethane:ethanol:0.880 ammonia; 400:8:1 gave the title compound as a colourless solid (42 mg).

¹H NMR (DMSO, δ) 5.40 (d, 1H) 7.27-7.70 (m, 12H) 8.22-8.26 (m, 2H) 8.72 (s, 1H) 8.88 (d, 1H) 11.14 (s, 1H)

RT=5.22, ES+ 423.49

### Example 52

### 1-(2-Oxo-5-phenyl-2,3-dihydro-1H-benzo[e][1,4]diazepin-3-yl)-3-(4-phenoxy-phenyl)-urea

Racemic 3-Amino-5-phenyl-1,3-dihydro-benzo[e][1,4]diazepin-2-one (30 mg) and 1-isocyanato-4-phenoxy-benzene (0.022 ml) in dry THF (4 ml) was stirred at room temperature for 18 h. The mixture was then partitioned between water and dichloromethane. The dried organic layer was evaporated and the residue triturated from dichloromethane/diethyl ether giving the title compound as a white solid (25 mg)

1H NMR (DMSO, d) 5.23 (d, 1H) 6.98-7.03 (m, 3H) 7.11 (t, 1H) 7.33-7.58 (m, 13H) 7.71 (dt, 1H) 9.18 (s, 1H) 11.03 (brs, 1H)

RT=5.57, ES+ 463.45

### Activity Example 1

Mouse monoclonal antibodies to the phosphoprotein (P), nucleocapsid (N) & fusion (F) proteins of RSV and a rabbit anti-mouse-horseradwash peroxidase (HRP) conjugated secondary antibody were used to demonstrate a reduction in RSV antigen via conversion of the o-phenylene diamine dihydrochloride (OPD) substrate to a coloured product. This was quantified by optical density (OD) measurement.

This assay was set up using all 96 wells of flat-bottomed 96-well plates. The outer wells were not subjected to any greater amount of evaporation than the inner wells during the 3 day assay period. (i.e. No “edge effect” seen).

Plates were set up one day before addition of virus and compounds. The assay then ran for 3 days with ELISA development taking place on the 4^{th} day.

### Day 0

### Set up of Assay Plates

All 96 wells of a microtitre plate were seeded at a density of 4×10³ Hep-2 cells/well in 100 μl/well of Growth Medium (GM) consisting of Dulbecco's MEM (DMEM) with Glutamax-1, Sodium Pyruvate, 1000 mg/l glucose and pyridoxine (Invitrogen, catalogue number 21885-025) and supplemented with 10% FBS. (See Plate 1).

In tissue culture, the cells adhere to the tissue culture flask and were grown at 37° C., 5% CO₂ until 90% confluent.

Monolayers were washed with 20 ml sterile PBS to remove serum and treated with 1 ml trypsin to detach cells from the flask.

Cells were suspended in a small known volume of growth media and counted using a haemocytometer. The cell suspension was made up to the desired concentration in growth medium and added to wells by multichannel pipette. Brief, gentle shaking encouraged the cells to disperse more evenly across the well.

Plate 1

| | | | | | | | | | | | |
|---|---|---|---|---|---|---|---|---|---|---|---|
| cells | cells | cells | cells | cells | cells | cells | cells | cells | cells | cells | cells |
| cells | cells | cells | cells | cells | cells | cells | cells | cells | cells | cells | cells |
| cells | cells | cells | cells | cells | cells | cells | cells | cells | cells | cells | cells |
| cells | cells | cells | cells | cells | cells | cells | cells | cells | cells | cells | cells |
| cells | cells | cells | cells | cells | cells | cells | cells | cells | cells | cells | cells |
| cells | cells | cells | cells | cells | cells | cells | cells | cells | cells | cells | cells |
| cells | cells | cells | cells | cells | cells | cells | cells | cells | cells | cells | cells |
| cells | cells | cells | cells | cells | cells | cells | cells | cells | cells | cells | cells |

Plates were kept undisturbed at 37° C. in a 5% CO₂ atmosphere for 24 hrs during which time the cells settle to form an even cell monolayer.

### Day 1

### Addition of Virus

A frozen vial of RSV (RSS strain provided by Virogen Ltd) stock solution was removed from the −80 freezer or liquid nitrogen store and diluted to a known Multiplicity of Infection (m.o.i) in Growth Medium.

The m.o.i. was calculated by prior titration of the virus stock (by the ELISA assay method) as the virus input required to achieve a window of at least 0.8 OD units between infected and uninfected control wells.

$$Multiplicity of Infection = \frac{plaque forming units per well ( pfu \text{/} well )}{number of cells per well}$$

50 μl of diluted virus was added to infected, “virus+”, wells by multichannel pipette; 50 μl of Growth Medium was added to uninfected, cell control wells (CC) by multichannel pipette. (See Plate 2)

Plate 2

| | | | | | | | | | | | |
|---|---|---|---|---|---|---|---|---|---|---|---|
| virus+ | virus+ | virus+ | virus+ | virus+ | virus+ | virus+ | virus+ | virus+ | virus+ | virus+ | virus+ |
| virus+ | virus+ | virus+ | virus+ | virus+ | virus+ | virus+ | virus+ | virus+ | virus+ | virus+ | virus+ |
| virus+ | virus+ | virus+ | virus+ | virus+ | virus+ | virus+ | virus+ | virus+ | virus+ | virus+ | virus+ |
| virus+ | virus+ | virus+ | virus+ | virus+ | virus+ | virus+ | virus+ | virus+ | virus+ | virus+ | virus+ |
| virus+ | virus+ | virus+ | virus+ | virus+ | virus+ | virus+ | virus+ | virus+ | virus+ | virus+ | virus+ |
| virus+ | virus+ | virus+ | virus+ | virus+ | virus+ | virus+ | virus+ | virus+ | virus+ | virus+ | virus+ |
| virus+ | virus+ | virus+ | virus+ | virus+ | virus+ | virus+ | virus+ | virus+ | virus+ | virus+ | virus+ |
| virus+ | virus+ | virus+ | virus+ | virus+ | virus+ | CC | CC | CC | CC | CC | CC |

Sides of plates were marked with stripes to identify plates in the event of lids becoming separated.

Plates were incubated at 37° C. for 1 hr to allow virus adsorption.

### Compound Dilutions

Compounds were made up at 4× strength in GM containing 2% DMSO (a final DMSO concentration in the assay of 0.5%).

Six compounds were tested on each assay plate as illustrated below. (See Plate 3). Compounds were tested in duplicate wells across a 7-point dilution series (from 50 μM-0.78 μM): in the presence of virus.

Virus infected, untreated wells served as the virus control (VC); Uninfected, untreated wells serve as the cell control (CC). The difference in absorbance between CC and VC wells constitutes the assay window.

Plate 3

| | | | | | | | | | | | | | |
|---|---|---|---|---|---|---|---|---|---|---|---|---|---|
| 50 | μM | Cpd1 | Cpd1 | Cpd2 | Cpd2 | Cpd3 | Cpd3 | Cpd4 | Cpd4 | Cpd5 | Cpd5 | Cpd6 | Cpd6 |
| 25 | μM | Cpd1 | Cpd1 | Cpd2 | Cpd2 | Cpd3 | Cpd3 | Cpd4 | Cpd4 | Cpd5 | Cpd5 | Cpd6 | Cpd6 |
| 12.5 | μM | Cpd1 | Cpd1 | Cpd2 | Cpd2 | Cpd3 | Cpd3 | Cpd4 | Cpd4 | Cpd5 | Cpd5 | Cpd6 | Cpd6 |
| 6.25 | μM | Cpd1 | Cpd1 | Cpd2 | Cpd2 | Cpd3 | Cpd3 | Cpd4 | Cpd4 | Cpd5 | Cpd5 | Cpd6 | Cpd6 |
| 3.125 | μM | Cpd1 | Cpd1 | Cpd2 | Cpd2 | Cpd3 | Cpd3 | Cpd4 | Cpd4 | Cpd5 | Cpd5 | Cpd6 | Cpd6 |
| 1.56 | μM | Cpd1 | Cpd1 | Cpd2 | Cpd2 | Cpd3 | Cpd3 | Cpd4 | Cpd4 | Cpd5 | Cpd5 | Cpd6 | Cpd6 |
| 0.78 | μM | Cpd1 | Cpd1 | Cpd2 | Cpd2 | Cpd3 | Cpd3 | Cpd4 | Cpd4 | Cpd5 | Cpd5 | Cpd6 | Cpd6 |
| 0 | μM | VC | VC | VC | VC | VC | VC | CC | CC | CC | CC | CC | CC |

### Dilution Plate Set Up

Compounds were serially diluted out in a separate microtitre plate as follows. (See Plate 4)

200 μl of GM containing 2% DMSO was added to all wells except the ‘50 μM’ or first column, to which 392 μl of GM was added. 8 μl of each test compound was cherry-picked from a thawed Arrow screening plate and transferred to the appropriate well in the ‘50 μM’ column. Since the compound stock was at 10 mM in 100% DMSO, this will maintain the DMSO level at 2% at the top compound concentration.

Using a multichannel pipette, 200 μl was transferred from the 50 μM column to the 25 μM column, then to the 12.5 μM column and so on across the dilution plate creating a serial doubling dilution. Compounds were mixed upon transfer and tips changed between transfers, ensuring also that no compound was transferred to the last column of compound-free wells (0 μM).

Plate 4

| | 50 | 25 | 12.5 | 6.25 | 3.125 | 1.56 | 0.78 | 0 | | | | |
|---|---|---|---|---|---|---|---|---|---|---|---|---|
| | μM | μM | μM | μM | μM | μM | μM | μM | BL | BL | BL | BL |
| Cpd1 | 392 | 200 | 200 | 200 | 200 | 200 | 200 | 200 | BL | BL | BL | BL |
| Cpd2 | 392 | 200 | 200 | 200 | 200 | 200 | 200 | 200 | BL | BL | BL | BL |
| Cpd3 | 392 | 200 | 200 | 200 | 200 | 200 | 200 | 200 | BL | BL | BL | BL |
| Cpd4 | 392 | 200 | 200 | 200 | 200 | 200 | 200 | 200 | BL | BL | BL | BL |
| Cpd5 | 392 | 200 | 200 | 200 | 200 | 200 | 200 | 200 | BL | BL | BL | BL |
| Cpd6 | 392 | 200 | 200 | 200 | 200 | 200 | 200 | 200 | BL | BL | BL | BL |
| Cpd7 | 392 | 200 | 200 | 200 | 200 | 200 | 200 | 200 | BL | BL | BL | BL |
| Cpd8 | 392 | 200 | 200 | 200 | 200 | 200 | 200 | 200 | BL | BL | BL | BL |

BL = blank/empty well

### Addition of Compound

The dilution plate was turned lengthways and 50 μl of compound easily transferred by multichannel pipette from the dilution plate to the assay plate, column by column. There was therefore an excess of 100 μl remaining in the dilution plate.

Plates were incubated at 37° C., 5% CO₂ for 3 days.

### ELISA Stage

### Day 4

Media was tapped out from wells directly into Virkon (1% solution in water) and plates were washed by immersing in a plastic box containing PBS. 50 μl/well of 75%/25% vol/vol acetone/methanol fixative was added by multichannel pipette and left for 3 mins.

Acetone/methanol was discarded from wells into Virkon and wells were washed with PBS as above.

Some 200 μl of blocking solution (2% Marvel in PBS containing 0.05% Tween) was added per well by multichannel pipette. Plates were incubated at 37° C. in a shaking incubator for 60 mins.

Block solution was discarded down the sink and diluted primary antibody was added directly to wells (i.e. no washing required).

RSV mouse monoclonal antibody NCL-RSV3 (Novocastra) was diluted 1/400 in PBS/2% Marvel/0.05% Tween and 50 μl was added per well. Plates were incubated at 37° C. in a shaking incubator for 90 mins.

Antibody was discarded down the sink and plates were washed 4 times by immersion in PBS/0.05% Tween.

DAKo rabbit anti-mouse HRP conjugate (DAKO catalogue number P0260) was diluted 1/1000 in PBS/2% Marvel/0.05% Tween and 50 μl was added per well. Plates were incubated at 37° C. in a shaking incubator for 60 mins.

Antibody was discarded down the sink and plates were washed 6 times by immersion in PBS/0.05% Tween.

Substrate (SigmaFast OPD) was prepared in advance by dissolving 1 urea tablet in 20 mL water. 1 OPD tablet was added to the urea solution just prior to use (NB. OPD was light sensitive) and vortexed to mix. 50 μl of substrate was added per well.

The reaction was stopped by addition of 25 μl/well of 20% sulphuric acid, once sufficient colour had developed but while cell control background was still low (˜5 minutes).

Plates were read on a SpectraMax (Molecular Devices) spectrophotometer at wavelength 490 nm and utilize the SOFTmax Pro software package.

The wells were emptied, washed in tap water and the monolayers stained with 50 μl/well of 2% crystal violet in 20% methanol/water for at least 1 hour. The wells were then washed and air-dried and the monolayers examined under the microscope for indications of cell toxicity.

### Results

SOFTmax data files were exported to Excel. Data handling used Excel templates written in-house for plotting dose response curves graphically and calculating IC50 values from the curves obtained.

All replicate wells were meaned. The assay window was calculated by subtracting the meaned cell control (CC) from the meaned virus control (VC). For each compound, the meaned CC was subtracted from the meaned values for each concentration point. The % of control was then calculated for each concentration point as a percentage of the window.

% of control was plotted against compound concentration. A straight line was fitted to the curve and the slope and intercept functions were used to calculate the IC50.

| Example | | |
|---|---|---|
| Number | IC50 | TD50 |
| 1 | B | >50 |
| 2 | B | >50 |
| 3 | B | >50 |
| 4 | A | >50 |
| 5 | A | Tr25 |
| 6 | B | >50 |
| 7 | A | Tr50 |
| 8 | A | >50 |
| 9 | B | >50 |
| 10 | A | >50 |
| 11 | A | >50 |
| 12 | B | >50 |
| 13 | B | 12.5 |
| 14 | A | Tr50 |
| 15 | C | Tr25 |
| 16 | B | 12.5 |
| 17 | B | 12.5 |
| 18 | A | 6.25 |
| 19 | A | 25 |
| 20 | C | >50 |
| 21 | B | Tr25 |
| 22 | C | >25 |
| 23 | A | >50 |
| 24 | A | >50 |
| 25 | C | >50 |
| 26 | A | >50 |
| 27 | C | >50 |
| 28 | C | >50 |
| 29 | A | >50 |
| 30 | B | >50 |
| 31 | B | >50 |
| 32 | A | >50 |
| 33 | B | >50 |
| 34 | A | >50 |
| 35 | A | >50 |
| 36 | A | >50 |
| 37 | A | >50 |
| 38 | A | >50 |
| 39 | A | >50 |
| 40 | B | >50 |
| 41 | A | >50 |
| 42 | A | >50 |
| 43 | A | >50 |
| 44 | B | >50 |
| 45 | A | >50 |
| 46 | B | >50 |
| 47 | A | >50 |
| 48 | A | >50 |
| 49 | A | >50 |
| 50 | A | >100 |
| 51 | A | >50 |
| 52 | B | >100 |

A <5 μM
B = 5-10 μM
C >10 μM

1. Use of a compound which is (a) a benzodiazepine derivative of the formula (I) or an N-oxide thereof or (b) a pharmaceutically acceptable salt thereof, in the manufacture of a medicament for use in treating or preventing an RSV infection 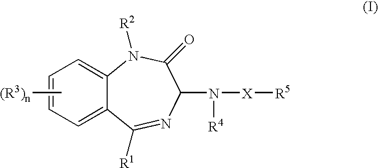
wherein:
R¹ represents C₁₋₆ alkyl, aryl or heteroaryl;
R² represents hydrogen or C₁₋₆ alkyl;
each R³ is the same or different and represents halogen, hydroxy, C₁₋₆ alkyl, C₁₋₆ alkoxy, C₁₋₆ alkylthio, C₁₋₆ haloalkyl, C₁₋₆ haloalkoxy, amino, mono(C₁₋₆ alkyl)amino, di(C₁₋₆ alkyl)amino, nitro, cyano, —CO₂R′, —CONR′R″, —NH—CO—R′, —S(O)R′, —S(O)₂R′, NH—S(O)₂R′, —S(O)NR′R″ or —S(O)₂NR′R″, wherein each R′ and R′ is the same or different and represents hydrogen or C₁₋₆ alkyl;
n is from 0 to 3;
R⁴ represents hydrogen or C₁₋₆ alkyl;
X represents —CO—, —CO—NR′—, —S(O)— or —S(O)₂—, wherein R′ is hydrogen or a C₁₋₆ alkyl group; and
R⁵ represents an aryl, heteroaryl or heterocyclyl group, which group is substituted by a C₁₋₆ hydroxyalkyl group or a —(C₁₋₄ alkyl)-X₁—(C₁₋₄ alkyl)-X₂—(C₁₋₄ alkyl) group, wherein X₁ represents —O—, —S— or —NR′—, wherein R′ represents H or a C₁₋₄ alkyl group and X₂ represents —CO—, —SO— or —SO₂—, or R₅ represents -A₁-Y-A₂, wherein:
A₁ is an aryl, heteroaryl, carbocyclyl or heterocyclyl group;
Y represents a direct bond or a C₁₋₄ alkylene, —SO₂—, —CO—, —O—, —S— or —NR′— moiety, wherein R′ is a C₁₋₆ alkyl group; and
A₂ is an aryl, heteroaryl, carbocyclyl or heterocyclyl group.

2. The use according to claim 1, wherein R¹ is C₁₋₂ alkyl or phenyl.

3. The use according to claim 1, wherein R² is hydrogen.

4. The use according to claim 1 wherein R³ is halogen, hydroxy, C₁₋₄ alkyl, C₁₋₄ alkoxy, C₁₋₄ alkylthio, C₁₋₄ haloalkyl, C₁₋₄ haloalkoxy, amino, mono(C₁₋₄ alkyl)amino or di(C₁₋₄ alkyl)amino.

5. The use according to claim 4, wherein R³ is fluorine, chlorine, bromine, C₁₋₂ alkyl, C₁₋₂ alkoxy, C₁₋₂ alkylthio, C₁₋₂ haloalkyl, C₁₋₂ haloalkoxy, amino, mono(C₁₋₂ alkyl)amino or di(C₁₋₂ alkyl)amino.

6. The use according claim 1 wherein R⁴ is hydrogen or C₁₋₂ alkyl.

7. The use according to claim 1 wherein X is —CO— or —CO—NR′— wherein R′ represents hydrogen or a C₁₋₂ alkyl group.

8. The use according to claim 1, wherein R⁵ is a 5- or 6-membered heterocyclyl or heteroaryl ring which is substituted by a C₁₋₆ hydroxyalkyl group or a —(C₁₋₄ alkyl)-X₁—(C₁₋₄ alkyl)-X₂—(C₁₋₄ alkyl) group, wherein X₁ and X₂ are as defined in claim 1.

9. The use according to claim 8, wherein R⁵ is a 5- or 6-membered heteroaryl group which is substituted by a —CH₂—OH or —(C₁₋₄ alkyl)-NR′—(C₁₋₄ alkyl)-S(O)₂—(C₁₋₄ alkyl) substituent, wherein R is hydrogen or C₁₋₂ alkyl.

10. The use according to claim 1, wherein A₁ is an aryl or heteroaryl group.

11. The use according to claim 10, wherein A₁ is a phenyl group, a monocyclic 5- or 6-membered heteroaryl group or a 5- to 6-membered heteroaryl group fused to a monocyclic oxo-substituted 5- to 6-membered heterocyclyl group.

12. The use according to claim 1 wherein A₁ is unsubstituted or substituted by 1 or 2 substituents selected from halogen, cyano, nitro, C₁₋₄ alkyl, C₁₋₄ haloalkyl and C₁₋₄ alkoxy substituents.

13. The use according to claim 1, wherein Y represents a direct bond, a C₁₋₂ alkylene group, —SO₂— or —O—.

14. The use according to claim 1, wherein A₂ is a phenyl, 5- to 6-membered heteroaryl, 5- to 6-membered heterocyclyl or C₃₋₆ cycloalkyl group.

15. The use according to claim 1 wherein when A₂ is a heterocyclyl group it is attached to the moiety Y via a N atom.

16. The use according to claim 1, wherein A₂ is unsubstituted or is substituted by 1 or 2 substituents which are selected from C₁₋₄ alkyl and halogen substituents when A₂ is a heteroaryl or aryl group and which are selected from C₁₋₄ alkyl, halogen and oxo substituents when A₂ is a carbocyclic or heterocyclyl group.

17. The use according to claim 1, wherein A₂ is a piperazinyl, pyridyl, morpholinyl, pyrrolidinyl, piperidinyl, pyrazinyl, cyclopropyl, phenyl or S,S-dioxo-thiomorpholino group, which is unsubstituted or substituted by a C₁₋₂ alkyl group.

18. The use according to claim 1 wherein the benzodiazepine derivative of formula (I) is a benzodiazepine derivative of formula (Ia): 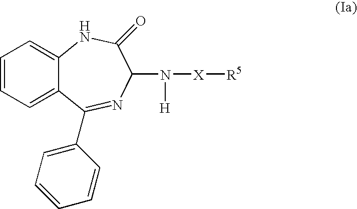
wherein:
X is —CO— or —CO—NH—; and
R⁵ is a 5- to 6-membered heteroaryl group, for example a furanyl group, which is substituted by —CH₂—OH or —(C₁₋₄ alkyl)-N(CH₃)—(C₁₋₄ alkyl)SO₂—(C₁₋₄ alkyl) or R⁵ represents -A₁-Y-A₂, wherein: A₁ is a phenyl, pyridyl, furanyl, thiazolyl, oxazolyl, isoxazolyl, thienyl or 1H-imidazo[4,5-b]pyridin-2-(3H)-one moiety, which is unsubstituted or substituted by 1 or 2 substituents selected from halogen, cyano, C₁₋₂ alkyl, C₁₋₂ haloalkyl and C₁₋₂ alkoxy substituents;
Y is a direct bond, a C₁₋₂ alkylene group, —SO₂— or —O—; and
A₂ is a piperazinyl, pyridyl, morpholinyl, pyrrolidinyl, piperidinyl, pyrazinyl, cyclopropyl, phenyl or S,S-dioxo-thiomorpholino group, which is unsubstituted or substituted by a C₁₋₂ alkyl group.

19. The use according to claim 1, wherein the medicament is for use in treating a patient who is a child under two years of age, an adult suffering from asthma, chronic obstructive pulmonary disorder (COPD) or immunodeficiency, an elderly person or a person in a long term care facility.

20. The use according to claim 19 wherein said child suffers from chronic lung disease.

21. The use according to claim 1 wherein the medicament is for use in preventing RSV infection in an infant less than six years of age who was born after 32 weeks of gestation or less.

22. The use according to claim 1, wherein the medicament is suitable for intranasal or intrabronchial administration.

23. The use according to claim 1 wherein the medicament further comprises an anti-inflammatory compound or an anti-influenza compound.

24. The use according to claim 23 wherein the anti-inflammatory compound is budesonide or fluticasone.

25. The use according to claim 23 wherein the anti-inflammatory compound is a leukotriene antagonist, phosphodiesterase 4 inhibitor or TNF alpha inhibitor.

26. The use according to claim 23 wherein the anti-inflammatory compound is an interleukin 8 or interleukin 9 inhibitor.

27. The use according to claim 1 wherein the medicament is coadministered with an anti-inflammatory compound, wherein the anti-inflammatory compound is selected from the group consisting of budesonide, fluticasone, a leukotriene antagonist, phosphodiesterase 4 inhibitor, TNF alpha inhibitor, an interleukin 8 inhibitor and an interleukin 9 inhibitor.

28. A method of treating a patient suffering from or susceptible to an RSV infection, which method comprises administering to said patient an effective amount of a compound as defined in claim 1.

29. A method according to claim 28, wherein said patient is a patient selected from the group consisting of a child under two years of age, an adult suffering from asthma, chronic obstructive pulmonary disorder (COPD) or immunodeficiency, an elderly person, a person in a long term care facility, a child under two years of age that suffers from chronic lung disease, and an infant less than six years of age who was born after 32 weeks of gestation or less.

30. A method according to claim 28, wherein said compound is administered intranasally or intrabronchially.

31. An inhaler or nebuliser containing a medicament which comprises
(a) a compound as defined in claim 1, and
(b) a pharmaceutically acceptable carrier or diluent.

32. A product comprising a compound as defined in claim 1 and an anti-inflammatory compound selected from the group consisting of budesonide, fluticasone, a leukotriene antagonist a phosphodiesterase 4 inhibitor, a TNF alpha inhibitor, an interleukin 8 inhibitor and an interleukin 9 inhibitor, or an anti-influenza compound.

33. The use of a product according to claim 32 in the manufacture of a medicament for use in the treatment of concomitant RSV and influenza infections.

34. The use of a compound as defined in claim 1 in the manufacture of a medicament for use in the treatment of human metapneumo virus, measles, parainfluenza viruses, mumps, yellow fever virus (B5 strain), Dengue 2 virus or West Nile virus.

35. A compound which is (a) a benzodiazepine derivative of formula (Ib) or an N-oxide thereof, or (b) a pharmaceutically acceptable salt thereof 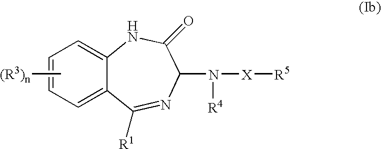
wherein R₁, R₃, n, R₄, X and R₅ are as defined in claim 1.

36. A compound according to claim 35, wherein R₁ is an unsubstituted phenyl group.

37. A compound according to claim 35, wherein when A₁ is a heteroaryl group, it is other than a 5-methyl-isoxazolyl moiety.

38. A compound according to claim 1, wherein A₁ is an aryl or heteroaryl moiety.

39. A compound according to claim 1, wherein X is —CO— or —CO—NR′—, wherein R′ is as defined in claim 1, provided that when X is —CO—NR′—, the moiety -A₁-Y-A₂ is -phenyl-O-phenyl.

40. A compound according to claim 1, wherein A₂ is other than a 4- to 10-membered saturated cycloalkyl ring, in which one of the carbon atoms is replaced by a N atom.

41. A compound according to claim 1, wherein A₂ is a piperazinyl, pyridyl, pyrrolidinyl, pyrazinyl, cyclopropyl, phenyl or S,S-dioxo-thiomorpholino group which is unsubstituted or is substituted by a C₁₋₂ alkyl group.

42. A compound according to claim 35, wherein the benzodiazepine derivative of the formula (Ib) is:
6-(4-Methyl-piperazin-1-yl)-N-(2-oxo-5-phenyl-2,3-dihydro-1H-benzo[e][1,4]diazepin-3-yl)-nicotinamide;
3,4,5,6-Tetrahydro-2H-[1,2′]bipyridinyl-5′-carboxylic acid (2-oxo-5-phenyl-2,3-dihydro-1H-benzo[e][1,4]diazepin-3-yl)-amide;
(S)-2-(1,1-Dioxo-1λ6-thiomorpholin-4-yl)-N-(2-oxo-5-phenyl-2,3-dihydro-1H-benzo[e][1,4]diazepin-3-yl-benzamide;
(S)-2-Chloro-4-morpholin-4-yl-N-(2-oxo-5-phenyl-2,3-dihydro-1H-benzo[e][1,4]diazepin-3-yl)-benzamide;
(S)-2-(1,1-Dioxo-1λ6-thiomorpholin-4-yl)-4-fluoro-(2-oxo-5-phenyl-2,3-dihydro-1H-benzo[e][1,4]diazepin-3-yl-benzamide;
(S)-5-Chloro-2-(1,1-dioxo-1λ6-thiomorpholin-4-yl)-N-(2-oxo-5-phenyl-2,3-dihydro-1H-benzo[e][1,4]diazepin-3-yl)-benzamide;
(S)-2-(1,1-Dioxo-1λ6-thiomorpholin-4-yl)-5-fluoro-N-(2-oxo-5-phenyl-2,3-dihydro-1H-benzo[e][1,4]diazepin-3-yl)-benzamide;
(S)-5-(4-Methyl-piperazin-1-ylmethyl)-furan-2-carboxylic acid (2-oxo-5-phenyl-2,3-dihydro-1H-benzo[e][1,4]diazepin-3-yl)-amide;
(S)-5-Pyrrolidin-1-ylmethyl-furan-2-carboxylic acid (2-oxo-5-phenyl-2,3-dihydro-1H-benzo[e][1,4]diazepin-3-yl)-amide;
(S)-5-Piperidin-1-ylmethyl-furan-2-carboxylic acid (2-oxo-5-phenyl-2,3-dihydro-1H-benzo[e][1,4]diazepin-3-yl)-amide;
(S)-5-Dimethylaminomethyl-furan-2-carboxylic acid (2-oxo-5-phenyl-2,3-dihydro-1H-benzo[e][1,4]diazepin-3-yl)-amide;
(S)-4-Fluoro-N-(2-oxo-5-phenyl-2,3-dihydro-1H-benzo[e][1,4]diazepin-3-yl)-2-piperidin-1-yl-benzamide;
(S)-4-Fluoro-2-morpholino-4-yl-N-(2-oxo-5-phenyl-2,3-dihydro-1H-benzo[e][1,4]diazepin-3-yl)-benzamide;
(S)-4-Cyano-N-(2-oxo-5-phenyl-2,3-dihydro-1H-benzo[e][1,4]diazepin-3-yl)-2-pyrrolidin-1-yl-benzamide,
(S)-4-Cyano-N-(2-oxo-5-phenyl-2,3-dihydro-1H-benzo[e][1,4]diazepin-3-yl)-piperidine-1-yl-benzamide;
(S)—N-(2-Oxo-5-phenyl-2,3-dihydro-1H-benzo[e][1,4]diazepin-3-yl)-2-pyrrolidin-1-yl-4-trifluoromethyl-benzamide;
(S)—N-(2-Oxo-5-phenyl-2,3-dihydro-1H-benzo[e][1,4]diazepin-3-yl)-2-piperidin-1-yl-4-trifluoromethyl-benzamide;
(S)-2-Morpholin-4-yl-N-(2-oxo-5-phenyl-2,3-dihydro-1H-benzo[e][1,4]diazepin-3-yl)-4-trifluoromethyl-benzamide;
(S)—N-(2-Oxo-5-phenyl-2,3-dihydro-1H-benzo[e][1,4]diazepin-3-yl)-2-pyrrolidin-1-yl-5-trifluoromethyl-benzamide;
(S)-2-Morpholin-4-yl-N-(2-oxo-5-phenyl-2,3-dihydro-1H-benzo[e][1,4]diazepin-3-yl)-5-trifluoromethyl-benzamide;
(S)-2-Morpholin-4-yl-N-(2-oxo-5-phenyl-2,3-dihydro-1H-benzo[e][1,4]diazepin-3-yl)-nicotinamide;
(S)-2-(1,1-Dioxo-1λ6-thiomorpholin-4-yl)-N-(2-oxo-5-phenyl-2,3-dihydro-1H-benzo[e][1,4]diazepin-3-yl)-nicotinamide;
(S)-2-(1,1-Dioxo-1λ6-thiomorpholin-4-yl)-3-methyl-N-(2-oxo-5-phenyl-2,3-dihydro-1H-benzo[e][1,4]diazepin-3-yl)-benzamide;
(S)-2-(1,1-Dioxo-1λ6-thiomorpholin-4-yl)-4-methyl-N-(2-oxo-5-phenyl-2,3-dihydro-1H-benzo[e][1,4]diazepin-3-yl)-benzamide;
(S)-2-(1,1-Dioxo-1λ6-thiomorpholin-4-yl)-6-methyl-N-(2-oxo-5-phenyl-2,3-dihydro-1H-benzo[e][1,4]diazepin-3-yl)-benzamide;
(S)-2-Chloro-6-(1,1-dioxo-1λ6-thiomorpholin-4-yl)-N-(2-oxo-5-phenyl-2,3-dihydro-1H-benzo[e][1,4]diazepin-3-yl)-benzamide;
(S)-3-Cyclopropyl-2-oxo-2,3-dihydro-imidazo[4,5-b]pyridine-1-carboxylic acid (2-oxo-5-phenyl-2,3-dihydro-1H-benzo[e][1,4]diazepin-3-yl)-amide;
(S)-3-(4-Methyl-piperazine-1-sulfonyl)-N-(2-oxo-5-phenyl-2,3-dihydro-1H-benzo[e][1,4]diazepin-3-yl)-benzamide;
(S)-4-(4-Methyl-piperazin-1-yl)-N-(2-oxo-5-phenyl-2,3-dihydro-1H-benzo[e][1,4]diazepin-3-yl)-benzamide;
(S)—N-(2-Oxo-5-phenyl-2,3-dihydro-1H-benzo[e][1,4]diazepin-3-yl)-3-(piperidine-1-sulfonyl)-benzamide;
(S)-3-(Morpholine-4-sulfonyl)-N-(2-oxo-5-phenyl-2,3-dihydro-1H-benzo[e][1,4]diazepin-3-yl)-benzamide;
(S)-5-Morpholin-4-ylmethyl-furan-2-carboxylic acid (2-oxo-5-phenyl-2,3-dihydro-1H-benzo[e][1,4]diazepin-3-yl)-amide;
(S)-5-Hydroxymethyl-furan-2-carboxylic acid (2-oxo-5-phenyl-2,3-dihydro-1H-benzo[e][1,4]diazepin-3-yl)-amide;
(S)-5-(1,1-Dioxo-1λ6-thiomorpholin-4-ylmethyl)-furan-2-carboxylic acid (2-oxo-5-phenyl-2,3-dihydro-1H-benzo[e][1,4]diazepin-3-yl)-amide;
(S)-2-Chloro-4-(1,1-dioxo-1λ6-thiomorpholin-4-yl)-N-(2-oxo-5-phenyl-2,3-dihydro-1H-benzo[e][1,4]diazepin-3-yl)-benzamide;
(S)-2-Chloro-5-(1,1-dioxo-1λ6-thiomorpholin-4-yl)-N-(2-oxo-5-phenyl-2,3-dihydro-1H-benzo[e][1,4]diazepin-3-yl)-benzamide;
(S)-5-{[(2-Methanesulfonyl-ethyl)-methyl-amino]-methyl}-furan-2-carboxylic acid (2-oxo-5-phenyl-2,3-dihydro-1H-benzo[e][1,4]diazepin-3-yl-amide;
(S)-2-Pyridin-3-yl-thiazole-4-carboxylic acid (2-oxo-5-phenyl-2,3-dihydro-1H-benzo[e][1,4]diazepin-3-yl)-amide;
(S)-2-Pyridin-4-yl-thiazole-4-carboxylic acid (2-oxo-5-phenyl-2,3-dihydro-1H-benzo[e][1,4]diazepin-3-yl)-amide;
(S)-4-Methyl-2-pyrazin-2-yl-thiazole-5-carboxylic acid (2-oxo-5-phenyl-2,3-dihydro-1H-benzo[e][1,4]diazepin-3-yl)-amide;
(S)-2-Morpholin-4-ylmethyl-furan-3-carboxylic acid (2-oxo-5-phenyl-2,3-dihydro-1H-benzo[e][1,4]diazepin-3-yl)-amide;
(S)-3-Morpholin-4-ylmethyl-N-(2-oxo-5-phenyl-2,3-dihydro-1H-benzo[e][1,4]diazepin-3-yl)-benzamide;
(S)-5-Morpholin-4-ylmethyl-isoxazole-3-carboxylic acid (2-oxo-5-phenyl-2,3-dihydro-1H-benzo[e][1,4]diazepin-3-yl)-amide;
(S)-3-Morpholin-4-ylmethyl-furan-2-carboxylic acid (2-oxo-5-phenyl-2,3-dihydro-1H-benzo[e][1,4]diazepin-3-yl)-amide;
(S)-5-Pyridin-2-yl-thiophene-2-carboxylic acid (2-oxo-5-phenyl-2,3-dihydro-1H-benzo[e][1,4]diazepin-3-yl)-amide;
(S)-2-Methyl-4-(morpholin-4-sulfonyl)-furan-3-carboxylic acid (2-oxo-5-phenyl-2,3-dihydro-1H-benzo[e][1,4]diazepin-3-yl)-amide;
(S)-6-Morpholin-4-yl-N-(2-oxo-5-phenyl-2,3-dihydro-1H-benzo[e][1,4]diazepin-3-yl)-nicotinamide;
(S)-3-Morpholin-4-ylmethyl-thiophene-2-carboxylic acid (2-oxo-5-phenyl-2,3-dihydro-1H-benzo[e][1,4]diazepin-3-yl)-amide;
(S)-5-Morpholin-4-ylmethyl-thiophene-2-carboxylic acid (2-oxo-5-phenyl-2,3-dihydro-1H-benzo[e][1,4]diazepin-3-yl)-amide;
2-Morpholin-4-yl-N-(2-oxo-5-phenyl-2,3-dihydro-1H-benzo[e][1,4]diazepin-3-yl)-benzamide;
(S)-5-Phenyl-oxazole-4-carboxylic acid (2-oxo-5-phenyl-2,3-dihydro-1H-benzo[e][1,4]diazepin-3-yl)-amide; or
1-(2-Oxo-5-phenyl-2,3-dihydro-1H-benzo[e][1,4]diazepin-3-yl)-3-(4-phenoxy-phenyl)-urea.

43. A compound according to claim 35 for use in a method of treating the human or animal body.

44. A pharmaceutical composition comprising a compound according to claim 35, and a pharmaceutically acceptable diluant or carrier.

45. A composition according to claim 44 comprising an optically active isomer of a compound according to claim 35.

46. A composition according to claim 44 which is in the form of a tablet, troche, lozenge, aqueous or oily suspension, dispersible powders or granules.
